(19) United States
(12) Patent Application Publication
(10) Pub. No.: US 2024/0091277 A1
(43) Pub. Date: Mar. 21, 2024

(54) Perioperative Innate Immune Priming in Cancer Therapy

(71) Applicant: Qu Biologics Inc., Burnaby (CA)

(72) Inventors: Harold David Gunn, Vancouver (CA); Shirin Kalyan, Burnaby (CA); Michael Alex Ander Kennedy, Burnaby (CA); Rebecca Ann Craufurd Auer, Burnaby (CA); Christiano Tanese De Souza, Ottawa (CA); Ting Ting Alice Lau, Ottawa (CA)

(21) Appl. No.: 18/283,313

(22) PCT Filed: Mar. 23, 2022
(86) PCT No.: PCT/CA2022/050437

§ 371 Date: Sep. 21, 2023

Related U.S. Application Data
(60) Provisional application No. 63/165,539, filed on Mar. 24, 2021.

(51) Int. Cl. A61K 35/74; A61K 47/34; A61K 47/38; A61P 35/00

(52) U.S. Cl. CPC A61K 35/74; A61K 47/34; A61K 47/38; A61P 35/00

(57) ABSTRACT

Therapeutic modalities are provided involving the use of specific repertoirs of PRR ligands to ameliorate immune dysregulation in a perioperative period. In effect, innate immune system signaling is provoked in the perioperative period so as to facilitate a targeted immune response following surgery. Subjects may accordingly be treated with immunogenic formulations tailored to a specific target tissue to treat a subject during the perioperative period surrounding surgery to remove a solid tumor, where the target tissue is the site of the tumor or a characteristic site of metastasis for the cancer.

### FIELD

Innovations are disclosed in the field of medical science, including the treatment of cancers, relating to the use of surgery in combination with neoadjuvant and/or adjuvant treatment with tissue-specific preparations that contain innate immunogens, such as microbial components.

### BACKGROUND

In vertebrates, an important aspect of immunological regulation involves the concerted activity of the innate immune system and the adaptive immune system. This concerted activity involves metabolic, enzymatic and molecular genetic changes within immune cells, orchestrating an elaborate system of cellular, cytokine and chemokine communication pathways mediating the coordinated activity of the disparate components of these complementary systems (see Iwasaki & Madzhitov, 2015, Nature Immunology 16:343-353; WO0209748; WO03051305; Turner et al., 2014, BBA-Molecular Cell Research 1843:11 2563-2582). An aspect of this coordinated activity underlies the recognition that ligands of the pattern recognition receptors (PRRs) of the innate immune system may be used as vaccine adjuvants to improve an adaptive immune response (see Maisonneuve et al., 2014, PNAS 111(34), 12294-9; WO2007035368), and particular repertoirs of PRR ligands may be formulated together as site specific immunomodulators that provoke a therapeutic immune response in a target tissue (see WO2017185180).

Immunological memory, involving the recognition of specific antigens by B and T cell receptors, is a long recognized and central feature of the adaptive immune system, and the basis for vaccine efficacy (see Nature Immunology, Focus on immunological memory: June 2011, Volume 12 No 6 pp 461-575). Innate immune memory is a more recently recognized and less well understood characteristic of the immune system (see Netea et al., 2015, Nature Immunology 16, 675-679; and Bordon, 2014, Nature Reviews Immunology 14, 713).

The immune system responds in complex ways to major surgery, and this response has particular significance in the context of primary cancer surgery (see Metzner et al., Nat Rev Clin Oncol. 2020 May; 17(5):313-326. doi: 10.1038/s41571-019-0319-9. Epub 2020 Feb. 17). In view of the potential complications associated with surgery, neoadjuvant and adjuvant cancer therapies have typically been avoided during the immediate perioperative period.

### SUMMARY

Aspects of the disclosed therapeutic modalities involve the use of an effective amount of an immunogenic composition to treat perioperative immune dysregulation in a subject. Treatments include perioperative cancer treatments, in which the immunogenic composition is administered before and/or after surgery during the perioperative period. Cancer treatments may accordingly involve neoadjuvant or adjuvant treatment in a mammalian subject where a cancer forms a tumor in a target tissue, and/or the cancer is characterized by a potential to metastasize to the target tissue, and surgery is carried out to remove the tumor. The perioperative period may for example be within 1, 2, 3, 4, 5, 6, 7, 8, 9, 10, 11 or 12 or more months, before and/or after, a surgery date, or alternatively 2 weeks, 1 week, 6 days, 5 days, 4 days, 3 days, 2 days or 1 day before and/or after, a surgery date.

The immunogenic composition may for example comprise an artificial repertoire of mammalian pattern recognition receptor (PRR) ligands that recapitulates at least a portion of a PRR agonist signature of a microbial mammalian pathogen, such as a pathogen that is pathogenic in the target tissue. The repertoire of mammalian PRR ligands may be formulated together in a therapeutic vehicle for combined presentation following administration to the mammalian subject. The composition may include components of the microbial mammalian pathogen that are ligands for a plurality of mammalian PRRs, for example at least 5, 6, 7, 8, 9, 10, 11, 12, 13, 14 or 15 distinct mammalian PRRs. The immunogenic composition may be adapted and administered so as to modulate an immune response in the target tissue, for example an immune response that is effective to treat residual disease and thereby treat the cancer, for example by modulating an innate immune response in the target tissue.

The compositions may be for use by administration in an amount effective in the perioperative period to: increase CD69 expression; reduce expression of CD96, increase expression of MHCII; increase myelopoiesis; increase neutrophil cell counts; increase expression of CCR2 chemokine receptor expression on immune cells; and/or increase FNγ expression. Point of care tests may accordingly be used to monitor the perioperative immune response, including any of the foregoing aspects of the perioperative immune response.

The therapeutic uses recited herein are reflected in corresponding methods of treatment, and vice versa.

Implementations of the present innovations may include one or more of the following features. The use where the PRR ligands are PRR agonists. The use where the immunogenic composition modulates an innate immune response in the target tissue. The use where the repertoire of mammalian pattern recognition receptors is an artificial repertoire and the portion of the PRR agonist signature is a distinct portion that is different from any native PRR ligand signature of the microbial mammalian pathogen. The use where the subject is a mouse, cat, dog, horse, rodent or human. The use where the therapeutic vehicle includes a microbial cell, a recombinant microbial cell, a cellular fraction of the recombinant microbial cell, a cellular fraction of the microbial cell, a bacterial outer membrane fraction, a bacterial inner membrane fraction, a pellet from a gradient centrifugation of microbial cell components, microbial chromosomal dna, a microparticle or a liposome, each including components of the microbial mammalian pathogen that provide the PRR agonists that together make up the repertoire of PRR agonists. The use where the recombinant microbe includes a recombinant gene encoding a component of at least one of the PRR agonists. The use where the therapeutic vehicle includes a whole killed or attenuated microbial cell or recombinant microbial cell.

SSI delivery systems are provided, comprising SSI formulations for use in an effective amount to treat a cancer in a mammalian subject, in which the cancer forms a tumor in a target tissue, and/or the cancer is characterized by a potential to metastasize to the target tissue. Delivery systems may be employed so that therapeutic compositions are deployed in conjunction with surgical removal of the tumor on a surgery date, wherein surgical removal leaves a surgical wound. The therapeutic composition may comprise PRR ligands of, or whole killed or attenuated cells of, microbial mammalian pathogens that are pathogenic in the target tissue. The delivery systems may be adapted for use on the surgery date applied to the surgical wound so as to mediate release of the composition and thereby modulate an immune response in the target tissue that is effective to treat residual disease and thereby treat the cancer. Delivery systems may include a staged-release matrix encasing the mammalian pathogen or PRR ligands, and the staged-release matrix may be adapted to release the antigens from the matrix in a plurality of successive temporally separated dosing stages after the delivery system is applied to the surgical wound on the surgery date, with a therapeutically effective alliquote of antigens being released at each dosing stage during a perioperative period. The staged-release matrix may for example be made up of a surface-eroding polymer, such as a polyanhydride and/or a poly(ortho ester), or a cellulose acetate phthalate complexed with Pluronic F-127.

### BRIEF DESCRIPTION OF THE DRAWINGS

FIG. 1 is a data plot showing results from a murine cancer metastasis model, with mice having undergone surgery in the right hand column for each category of treatment, illustrating the total number of lung metastases quantified 3 days post-surgery.

FIG. 2 is a data plot showing results from a murine cancer metastasis model, with mice having undergone surgery in the right hand column for each category of treatment, illustrating NK cell activation in the spleen quantified by cells expressing CD69, 3 days post surgery.

FIG. 3 is a data plot showing results from a murine cancer metastasis model, with mice having undergone surgery in the right hand column for each category of treatment, illustrating NK cell activation in the spleen was quantified by cells expressing CD96, 3 days post surgery.

FIG. 4 is a data plot showing results from a murine cancer metastasis model, with mice having undergone surgery in the right hand column for each category of treatment, illustrating monocyte/macrophage activation in the spleen quantified by cells expressing MHCII, 3 days post surgery.

FIG. 5 is a data plot showing results from a murine cancer metastasis model, with mice having undergone surgery in the right hand column for each category of treatment, illustrating neutrophil cell counts (CD11b+Ly6G+) from the spleen quantified as % of live cells, 3 days post surgery.

FIG. 6 is a data plot showing results from a murine cancer metastasis model, with mice having undergone surgery in the right hand column for each category of treatment, illustrating splenic monocyte/macrophage CCR2 receptor expression measured 3 days post-surgery.

FIG. 7 is a data plot showing results from a murine cancer metastasis model, with mice having undergone surgery in the right hand column for each category of treatment. Immune activation was quantified on Day 1 post-surgery by stimulating whole blood with a cytokine cocktail for 12 hours and measuring IFNγ levels.

FIG. 8 is a data plot showing results from a perioperative murine liver metastasis model, illustrating dramatic reducing in tumor burden (% of liver) with QBECO SSI treatment.

### DETAILED DESCRIPTION

In the following detailed description, various examples are set out of particular embodiments, together with experimental procedures that may be used to implement a wide variety of modifications and variations in the practice of the present invention. For clarity, a variety of technical terms are used herein in accordance with what is understood to be the commonly understood meaning, as reflected in definitions set out below.

An “immunogen” refers to a molecule, or a composition comprising the molecule, that is capable of eliciting an immune response by an organism's immune system. An “antigen” refers to a molecule that is capable of binding to the product of an immune response.

“Pathogenic” agents are agents, such as microbes, such as bacteria or viruses, which are known to cause infection in a host in nature, and in this sense, “pathogenic” is used in the context of the present invention to mean “naturally pathogenic”. Although a wide variety of microbes may be capable of causing infection under artificial conditions, such as artificial inoculations of a microbe into a tissue, the range of microbes that naturally cause infection is necessarily limited, and well established by medical practice.

An “infection” is the state or condition in which the body or a part of it is invaded by a pathogenic agent (e.g., a microbe, such as a bacterium) which, under favorable conditions, multiplies and produces effects that are injurious (Taber's Cyclopedic Medical Dictionary, 14th Ed., C. L. Thomas, Ed., F. A. Davis Company, PA, USA). An infection may not always be apparent clinically and may result in only localized cellular injury. Infections may remain subclinical, and temporary if the body's defensive mechanisms are effective. Infections may spread locally to become clinically apparent as an acute, a subacute, or a chronic clinical infection or disease state. A local infection may also become systemic when the pathogenic agent gains access to the lymphatic or vascular. Infection is usually accompanied by inflammation, but inflammation may occur without infection.

“Inflammation” is the characteristic tissue reaction to injury (marked by swelling, redness, heat, and pain), and includes the successive changes that occur in living tissue when it is injured. Infection and inflammation are different conditions, although one may arise from the other (Taber's Cyclopedic Medical Dictionary, supra). Accordingly, inflammation may occur without infection and infection may occur without inflammation (although inflammation typically results from infection by pathogenic bacteria or viruses). Inflammation is characterized by the following symptoms: redness (rubor), heat (calor), swelling (tumour), pain (dolor). Localized visible inflammation on the skin may be apparent from a combination of these symptoms, particularly redness at a site of administration.

Various subjects may be treated or assayed or sampled in accordance with alternative aspects of the invention. As used herein, a “subject” is an animal, for e.g., a vertebrate or a mammal. Accordingly, a subject may be a patient, e.g., a human, suffering from a disease or disorder amenable to treatment, such as a cancer, a proliferative cell disorder or infectious disease (such as a persistent viral infection or opportunistic fungal infection, particularly in an immunocompromised patient). A subject may also be an experimental animal, e.g., an animal model of an immune dysregulation. In some embodiments, the terms “subject” and “patient” may be used interchangeably, and may include a human, a non-human mammal, a non-human primate, a rat, mouse, cat or dog. A healthy subject may be a human who is not suffering from a disease, such as a cancer or immune dysfunction, or suspected of having the disease, or who is not suffering from a chronic disorder or condition. A “healthy subject” may also be a subject who is not immunocompromised. By immunocompromised is meant any condition in which the immune system functions in an abnormal or incomplete manner. Immunocompromisation may be due to disease, certain medications, or conditions present at birth. Immunocompromised subjects may be found more frequently among infants, the elderly, and individuals undergoing extensive drug or radiation therapy.

A “sample” from a subject may include any relevant biological material, including for example a cell, tissue or bodily fluid sample taken from a patient. For example, a sample may conveniently include samples of skin, cheek, blood, stool, hair or urine. Sample nucleic acids for use in diagnostic and prognostic methods can for example be obtained from a selected cell type or tissue of a subject. For example, a subject's bodily fluid (e.g. blood) can be obtained by known techniques. Alternatively, nucleic acid tests can be performed on dry samples (e.g., hair or skin).

Many of the methods described herein may be performed using kits, for example comprising at least one probe or primer nucleic acid, or one of more of the compositions described herein and instructions for use of the kit. Kits can for example comprise at least one probe or primer which is capable of specifically hybridizing to a polymorphic region or adjacent to the polymorphic region, so that the oligonucleotides are “specific for” the polymorphic region. Kits may also comprise at least one reagent necessary to perform a particular assay. Kits can also include positive controls, negative controls, sequencing markers, or antibodies, for example for determining a subject's genotype or biological marker profile.

An “immune response” includes, but is not limited to, one or more of the following responses in a mammal: induction of cellular immunomodulators such as cytokines and chemokines, induction or activation of antibodies, neutrophils, monocytes, macrophages (including both M1-like macrophages and M2-like macrophages as described herein), B cells, or T cells (including helper T cells, natural killer cells, cytotoxic T cells, gamma-delta (γδ) T cells), such as induction or activation by one or more immunogens in an immunogenic composition, following administration of the composition. An immune response to a composition thus generally includes the development in the host animal of a cellular and/or antibody-mediated response to the composition. In some embodiments, the immune response is such that it will also result in slowing or stopping the progression of an immune dysregulation, or a disease characterized by immune dysregulation. An immune response may accordingly include one or both of a cellular immune response and/or a humoral immune response, and may be an adaptive response or an innate immune response.

“Immune dysregulation” is an inappropriately regulated immune response, such as an inappropriately restrained or inappropriately robust immune response. The immune dysregulation may for example be in the context of a neoplastic disease, such as a cancer.

A “site specific immunotherapy” (SSI) is an immunomodulatory treatment that is effective to therapeutically or prophylactically alter an aspect of the immune state, or immune system physiology, at an anatomical site or sites, such as an organ or tissue. In some instances, for example, an SSI may be adapted to ameliorate an immune dysregulation, or to treat a condition characterized by an immune dysregulation.

A “cancer” or “neoplasm” is any unwanted growth of cells serving no physiological function. In general, a cancer cell has been released from its normal cell division control, i.e., a cell whose growth is not regulated by the ordinary biochemical and physical influences in the cellular environment. Thus, “cancer” is a general term for diseases characterized by abnormal uncontrolled cell growth. In most cases, a cancer cell proliferates to form clonal cells that are malignant. The lump or cell mass, “neoplasm” or “tumour,” is generally capable of invading and destroying surrounding normal tissues. By “malignancy”, as used herein, is meant as an abnormal growth of any cell type or tissue that has a deleterious effect in the organism having the abnormal growth. The term “malignancy” or “cancer” includes cell growths that are technically benign but which carry the risk of becoming malignant. Cancer cells may spread from their original site to other parts of the body through the lymphatic system or blood stream in a process known as “metastasis.” Many cancers are refractory to treatment and prove fatal. Examples of cancers or neoplasms include, without limitation, transformed and immortalized cells, tumours, carcinomas, in various organs and tissues as described herein or known to those of skill in the art.

Most cancers fall within three broad histological classifications: carcinomas, which are the predominant cancers and are cancers of epithelial cells or cells covering the external or internal surfaces of organs, glands, or other body structures (for e.g., skin, uterus, lung, breast, prostate, stomach, bowel), and which tend to metastasize; carcinomas, which are derived from connective or supportive tissue (for e.g., bone, cartilage, tendons, ligaments, fat, muscle); and hematologic tumours, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumours (mixed connective tissue types). Hematologic tumours may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumours and which develop in the glands or nodes of the lymphatic system, and which may be Hodgkin or Non-Hodgkin lymphomas. In addition, mixed type cancers, such as adenosquamous carcinomas, mixed mesodermal tumours, carcinosarcomas, or teratocarcinomas also exist.

Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas. Lymphomas may arise in the lymph nodes associated with the head, neck and chest, as well as in the abdominal lymph nodes or in the axillary or inguinal lymph nodes. Identification and classification of types and stages of cancers may be performed by using for example information provided by the Surveillance, Epidemiology, and End Results (SEER) Program of the National Cancer Institute, which is an authoritative source of information on cancer incidence and survival in the United States and is recognized around the world. The SEER Program currently collects and publishes cancer incidence and survival data from 14 population-based cancer registries and three supplemental registries covering approximately 26 percent of the US population. The program routinely collects data on patient demographics, primary tumour site, morphology, stage at diagnosis, first course of treatment, and follow-up for vital status, and is the only comprehensive source of population-based information in the United States that includes stage of cancer at the time of diagnosis and survival rates within each stage. Information on more than 3 million in situ and invasive cancer cases is included in the SEER database, and approximately 170,000 new cases are added each year within the SEER coverage areas. The incidence and survival data of the SEER Program may be used to access standard survival for a particular cancer site and stage. For example, to ensure an optimal comparison group, specific criteria may be selected from the database, including date of diagnosis and exact stage (for example, in the case of the lung cancer example herein, the years were selected to match the time-frame of the retrospective review, and stage 3B and 4 lung cancer were selected; and in the case of the colon cancer example herein, the years were also selected to match the time-frame of the retrospective review, and the stage 4 colon cancer was selected).

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body that is different from the primary site. With the present invention, treatment is directed to the site of the cancer, not type of cancer, so that a cancer of any type that is symptomatic or etiologically located in the lung, for example, would be treated on the basis of this localization in the lung.

Aspects of the invention relate to the use of PRR ligands. PRR ligands may for example be available commercially, for example in widely available preparations of attenuated or killed recombinant bacteria, which may for example be ligands for TLR2, TLR4 and TLR5. Compositions of pathogen-associated molecular patterns (PAMPs) may include PAMPS that are recognized by PRRs, including: Toll-like receptors (TLRs), NOD-like receptors (NLRs), RIG-I-like receptors (RLRs), C-type lectin receptors (CLRs) including Dectin-1, cytosolic dsDNA sensors (CDSs) and NLRs involved in the formation of inflammasomes.

Toll-like receptor 2 (TLR2) is involved in the recognition of a wide array of microbial molecules representing broad groups of species including Gram-positive and Gram-negative bacteria, as well as mycoplasma and yeast. TLR2 recognizes cell-wall components such as peptidoglycan, lipoteichoic acid and lipoprotein from Gram-positive bacteria, lipoarabinomannan from mycobacteria, and zymosan from the yeast cell wall. Toll-like receptor 3 (TLR3) recognizes double-stranded RNA (dsRNA). Bacterial lipopolysaccharide (LPS) is recognized by Toll-like receptor 4 (TLR4) which interacts with at least three different extracellular proteins: LPS-binding protein (LBP), CD14 and, myeloid differentiation protein 2 (MD-2), to induce a signaling cascade leading to the activation of NF-κB and the production of proinflammatory cytokines. LPS generally consists of a polysaccharide region that is anchored in the outer bacterial membrane by a carbohydrate lipid moiety: lipid A, which is largely responsible for the immunostimulatory activity of LPS. Particularly active forms of lipid A contain six fatty acyl groups, as for example may be found in pathogenic bacteria that are strains of Escherichia coli or Salmonella spp. Toll-like receptor 5 (TLR5) recognizes flagellin from both Gram-positive and Gram-negative bacteria. Toll-like receptor 7 (TLR7) and TLR8 recognize single stranded RNAs and small synthetic molecules such as imidazoquinolines and nucleoside analogs. Toll-like receptor 9 (TLR9) recognizes specific unmethylated CpG motifs prevalent in microbial but not vertebrate genomic DNA.

NLRs are a family of at least 22 cytoplasmic innate immune sensors, including NOD1 (CARD4) and NOD2 (CARD15) which are intracellular pattern-recognition receptors involved in the recognition of peptidoglycan (PGN). These receptors detect specific motifs within PGN. NOD1 senses the diaminopimelatic acid (DAP)-containing muropeptide (specifically d-Glu-meso-DAP dipeptide “iE-DAP” dipeptide) which is found primarily in PGN of Gram-negative bacteria, as well as certain Gram-positive bacteria. NOD2 recognizes the muramyl dipeptide (MDP) structure found in almost all bacterial PGN.

The RIG-I-Like receptors (RLRs), particularly RIG-I and MDA-5, detect viral RNA species.

CLR ligands include Dectin-1 and Mincle (macrophage-inducible C-type lectin) agonists. Dectin-1 is a specific receptor for 8-glucans, which are glucose polymers found in the cell walls of fungi. Mincle is a multi-tasking danger signal receptor that recognizes a wide variety of ligands such as damaged cells, fungal components, yeast components and components of mycobacteria.

Cytosolic DNA Sensors (CDS) bind intracellular DNA from pathogens, and there are multiple CDSs which may display contextual preferences for the recognition of particular DNAs.

Cyclic dinucleotides (CDNs) and xanthenone derivatives, such as DMXAA, bind to and activate STING (STimulator of INterferon Genes).

The inflammasome is a multi-protein complex involved in the production of mature IL-18, specifically through cleavage of pro-IL-18 and pro-IL-18 into active and secretable forms. Inflammasomes may be segregated into NLRP1, NLRP3, NLRC4 and AIM2 subtypes, which are activated by a wide variety of microbial molecules, danger signals and crystalline substances.

TABLE 1
PRR Receptors and their Ligands

| PRR | Ligand |
|---|---|
| TLR2 | Microbial cell wall |
| | components/preparations, Pam2C- |
| | Aca-Benzyl-Murabutide (Pam2C- |
| | conjugated murabutide) |
| TLR3 | Polyadenylic-polyuridylic acid, |
| | Polyinosine-polycytidylic acid |
| TLR4 | Lipopolysaccharide, Monophosphoryl |
| | Lipid A |
| TLR5 | Flagellin |
| TLR7/8 | Single-stranded RNAs, Nucleoside |
| | analogs, |
| | Imidazoquinolines/Thiazoquinolines |
| TLR9 | unmethylated CpG DNA motifs |
| NOD1 | iE-DAP, Acylated iE-DAP, D-gamma- |
| | Glu-mDAP, L-Ala-gamma-D-Glu- |
| | mDAP |
| NOD2 | MDP (MurNAc-L-Ala-D-isoGln, |
| | muramyl dipeptide), N-glycolylated |
| | muramyldipeptide, N-Acetyl-muramyl- |
| | L-Alanyl-D-Glutamin-n-butyl-ester, |
| | MurNAc-Ala-D-isoGln-Lys, N- |
| | Acetylmuramyl-L-Alanyl-D- |
| | Isoglutamine (L-D isoform), 6-O- |
| | stearoyl-N-Acetyl-muramyl-L-alanyl-D- |
| | isoglutamine, Pam2C-Aca-Benzyl- |
| | Murabutide, |
| TLR2/NOD2 | Pam2C-conjugated murabutide |
| NOD1/NOD2 | PGN, Pam2C-conjugated murabutide |
| RIG1/MDA5 | 5′ triphosphate double stranded RNA |
| | (18-20mer), |
| | polyriboinosinic:polyribocytidylic acid |
| DAI, LRRFIP1, AIM2, RIG1 | dsDNA, poly(dA-dT)•poly(dT-dA) |
| Dectin-1 | Beta-glucan peptide, fungal cell wall |
| | preparations |
| Mincle | damaged microbial cells, fungus, |
| | yeast and mycobacteria, Trehalose- |
| | 6,6-dibehenate, trehalose-6,6- |
| | dimycolate |
| STING | Cyclic dinucleotides (c-di-nucleotides), |
| | xanthenone derivatives, 3′3′-cGAMP, |
| | 2′3′-cGAMP, 2′2′-cGAMP, 2′2′- |
| | cGAMP, c-di-AMP (cyclic di-adenylate |
| | monophosphate), c-di-GMP, c-di-IMP, |
| | c-di-UMP, c-di-AMP |

TABLE 2
Cytosolic nucleic acid-sensing PRRs and their Ligands (Broz
& Monack, 2013, Nature Reviews Immunology 13, 551-565).

| PRR | Ligands |
|---|---|
| RIG-I | PPP-ssRNA (PPP-ssRNA, ssRNA with a 5′- |
| | triphosphate group), RNA with base pairing |
| | and polyl:C |
| MDA5 | Long dsRNA |
| LGP2 | dsRNA |
| DDX41 | B-form DNA and CDNs (cyclic dinucleotides) |
| DHX9 | DNA, RNA, CpG-A oligodeoxynucleotids and CpG-B |
| | ODNs |
| DDX3 | Viral RNA |
| DHX36 | DNA, RNA, CpG-A oligodeoxynucleotids and CpG-B |
| | oligodeoxynucleotids |
| DDX1-DDX21- | RNA and polyl:C |
| DDX36 | |
| DDX60 | ssRNA, dsRNA and dsDNA |
| KU70 | DNA |
| cGAS | DNA |
| STING | CDNs (c-di-GMP and c-di-AMP) |
| NOD2 | ssRNA |
| NLRP3 | ssRNA, dsRNA, bacterial mRNA and oxidized |
| | mitochondrial DNA |
| AIM2 | DNA |
| IFI16 | dsDNA |
| LRRFIP1 | B-form DNA, Z-form DNA and dsRNA |
| DAI | DNA |
| IFIT1, 2, 3 and 5 | PPP-ssRNA |

Aspects of the invention accordingly involve using PRR agonists derived from a selected microbial pathogen. For example, peptidoglycan (PGN) may be obtained from a bacteria or bacterial strain that is pathogenic in a selected target tissue or organ, for use as a NOD1/NOD2 agonist. Similarly, cell wall components may be obtained from a bacteria or bacterial strain that is pathogenic in a selected target tissue or organ, for use as a TLR2 agonist. Similarly, DNA, including double stranded DNA, particularly repetitive double stranded DNA, may be obtained from a microbial pathogen, such as a bacteria or bacterial strain that is pathogenic in a selected target tissue or organ, for use as a DAI, LRRFIP1, RIG1, TLR9, AIM2 or cytosolic DNA sensor (CDS) agonist. Beta-glucan peptides may be obtained from fungi or yeast that are pathogenic in a selected target tissue or organ, for use as a Dectin-1 agonists. Cyclic dinucleotides may be obtained from a microbial pathogen that is pathogenic in a selected target tissue or organ, for use as a STING agonist.

Aspects of the invention involve compositions that have a distinct PRR agonist signature, which connotes a repertoire of PRR agonists that are together collected in a therapeutic vehicle, so that the selected collection of PRR agonists is distinct. A “therapeutic vehicle” in this context is a formulation that aggregates and retains the PRR agonists, for example in a pharmaceutically acceptable particle or vesicle, such as a recombinant microbe. For example, the PRR agonist signature may be different from a reference PRR agonist signature, for example different from the collection of PRR agonists that would be present on a microbe that is not pathogenic in the target tissue. The PRR signature may also be distinct in the sense that it is different than a native PRR agonist signature of the microbial mammalian pathogen, for example altered by way of the recombinant expression of genes that alter what would otherwise be the wildtype PRR agonist signature of the pathogen. For purposes of determining the distinctiveness of a PRR agonist signature, the levels or kinds of PRR agonist may be directly measured, or may be measured for example by determining the activation or inhibition of a signaling pathway in a cell consequent to PRR agonist/receptor binding.

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. Nucleic acid “constructs” are accordingly recombinant nucleic acids, which have been generally been made by aggregating interoperable component sequencers. The term “recombinant” when made in reference to a protein or a polypeptide refers to a protein or polypeptide molecule which is expressed using a recombinant nucleic acid construct created by means of molecular biological techniques. The term “recombinant” when made in reference to the genetic composition or an organism or cell refers to new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention (so that it is anthropogenic). Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

Recombinant constructs of the invention may include a variety of functional molecular or genomic components, as required for example to mediate gene expression or suppression in a transformed plant. In this context, “DNA regulatory sequences,” “control elements,” and “regulatory elements,” refer to transcriptional and translational control sequences, such as promoters, enhancers, polyadenylation signals, terminators, and protein degradation signals that regulate gene expression, as well as epigenetic regulatory signals for example involving methylation or acetylation of histones (e.g. histone methyltransferase or acetyltransferase), leading to conformational changes in the transcriptional landscape and gene expression differences. In the context of the present disclosure, “promoter” means a sequence sufficient to direct transcription of a gene when the promoter is operably linked to the gene. The promoter is accordingly the portion of a gene containing DNA sequences that provide for the binding of RNA polymerase and initiation of transcription. Promoter sequences are commonly, but not universally, located in the 5′ non-coding regions of a gene. A promoter and a gene are “operably linked” when such sequences are functionally connected so as to permit gene expression mediated by the promoter. The term “operably linked” accordingly indicates that DNA segments are arranged so that they function in concert for their intended purposes, such as initiating transcription in the promoter to proceed through the coding segment of a gene to a terminator portion of the gene. Gene expression may occur in some instances when appropriate molecules (such as transcriptional activator proteins) are bound to the promoter. Expression is the process of conversion of the information of a coding sequence of a gene into mRNA by transcription and subsequently into polypeptide (protein) by translation, as a result of which the protein is said to be expressed. As the term is used herein, a gene or nucleic acid is “expressible” if it is capable of expression under appropriate conditions in a particular host cell.

An “isolated” nucleic acid or polynucleotide as used herein refers to a component that is removed from its original environment (for example, its natural environment if it is naturally occurring). An isolated nucleic acid or polypeptide may contain less than about 50%, less than about 75%, less than about 90%, less than about 99.9% or less than any integer value between 50 and 99.9% of the cellular or biological components with which it was originally associated. A polynucleotide amplified using PCR so that it is sufficiently distinguishable (on a gel from example) from the rest of the cellular components is, for example, thereby “isolated”. The polynucleotides of the invention may be “substantially pure,” i.e., having the high degree of isolation as achieved using a purification technique.

In the context of biological molecules “endogenous” refers to a molecule such as a nucleic acid that is naturally found in and/or produced by a given organism or cell. An “endogenous” molecule may also be referred to as a “native” molecule. Conversely, in the context of biological molecules “exogenous” refers to a molecule, such as a nucleic acid, that is not normally or naturally found in and/or produced by a given organism or cell in nature.

As used herein to describe nucleic acid or amino acid sequences, the term “heterologous” refers to molecules or portions of molecules, such as DNA sequences, that are artificially introduced into a particular host cell, for example by transformation. Heterologous DNA sequences may for example be introduced into a host cell by transformation. Such heterologous molecules may include sequences derived from the host cell. Heterologous DNA sequences may become integrated into the host cell genome, either as a result of the original transformation of the host cells, or as the result of subsequent recombination events.

Various aspects of the present disclosure encompass nucleic acid or amino acid sequences that are homologous to other sequences. As the term is used herein, an amino acid or nucleic acid sequence is “homologous” to another sequence if the two sequences are substantially identical and the functional activity of the sequences is conserved (as used herein, sequence conservation or identity does not infer evolutionary relatedness). Nucleic acid sequences may also be homologous if they encode substantially identical amino acid sequences, even if the nucleic acid sequences are not themselves substantially identical, for example as a result of the degeneracy of the genetic code.

PRR ligands for use in alternative aspects of the invention may be derived from microorganisms. More particularly, the microorganism that is the source of the PRR ligands may be pathogenic is a target tissue of interest. The characterization of a microbe as a pathogen is nuanced, in that most animals are colonized to some degree by microorganisms, such as bacteria, which exist in symbiotic or commensal relationships with the host animal. Thus, many species of normally harmless bacteria are found in healthy animals, and are usually localized to the surface of specific organs and tissues. Often, these microbial communities aid in the normal functioning of the body, as members of what is termed the microbiome. Microbes that are generally harmless, such as Escherichia coli, can cause infection in healthy subjects, with results ranging from mild infection to death. Whether or not a microorganism is pathogenic (i.e., causes infection) depends on factors such as: the route of entry and access to specific host cells, tissues, or organs; the intrinsic virulence of the microorganism; the amount of the microorganism present at the site of potential infection; or the health of the host animal. Thus, microorganisms that are normally harmless can become pathogenic given favorable conditions for infection, and even the most virulent microorganism generally requires specific circumstances to cause infection. Accordingly, microbial species that are members of the normal flora can be pathogens when they move beyond their normal ecological role in the endogenous flora. For example, endogenous species can cause infection outside of their ecological niche in regions of anatomical proximity, for example by contiguous spread. When this occurs, these normally harmless endogenous bacteria are pathogenic.

Specific microbial species are known to cause infections in specific cells, tissues, or organs in otherwise healthy subjects. Examples of bacteria and viruses that commonly cause infections in specific organs and tissues of the body are listed below; and these examples are not limiting in the sense that a skilled person would be able to recognize and identify infectious or pathogenic bacteria that cause infections, or commonly cause infections, in various organs and tissues in otherwise healthy organisms (and recognize the relative frequency of infection with each bacterial species) based on the knowledge in the field as represented, for example, by the following publications: Manual of Clinical Microbiology 8th Edition, Patrick Murray, Ed., 2003, ASM Press American Society for Microbiology, Washington DC, USA; Mandell, Douglas, and Bennett's Principles and Practice of Infectious Diseases 5th Edition, G. L. Mandell, J. E. Bennett, R. Dolin, Eds., 2000, Churchill Livingstone, Philadelphia, PA, USA, all of which are incorporated by reference herein.

Infections of the skin are commonly caused by the following bacterial species: Staphylococcus aureus, Beta hemolytic streptococci group A, B, C or G, Corynebacterium diptheriae, Corynebacterium ulcerans, or Pseudomonas aeruginosa; or viral pathogens: rubeola, rubella, varicella-zoster, echoviruses, coxsackieviruses, adenovirus, vaccinia, herpes simplex, or parvo B19.

Infections of the soft tissue (e.g., fat and muscle) are commonly caused by the following bacterial species: Streptococcus pyogenes, Staphylococcus aureus, Clostridium perfringens, or other Clostridium spp.; or viral pathogens: influenza, or coxsackieviruses.

Infections of the breast are commonly caused by the following bacterial species: Staphylococcus aureus, or Streptococcus pyogenes.

Infections of the lymph nodes of the head and neck are commonly caused by the following bacterial species: Staphylococcus aureus, or Streptococcus pyogenes; or viral pathogens: Epstein-Barr, cytomegalovirus, adenovirus, measles, rubella, herpes simplex, coxsackieviruses, or varicella-zoster.

Infections of the lymph nodes of the arm/axillae are commonly caused by the following bacterial species: Staphylococcus aureus, or Streptococcus pyogenes; or viral pathogens: measles, rubella, Epstein-Barr, cytomegalovirus, adenovirus, or varicella-zoster.

Infections of the lymph nodes of the mediastinum are commonly caused by the following bacterial species: viridans streptococci, Peptococcus spp., Peptostreptococcus spp., Bacteroides spp., Fusobacterium spp., or Mycobacterium tuberculosis; or viral pathogens: measles, rubella, Epstein-Barr, cytomegalovirus, varicella-zoster, or adenovirus.

Infections of the pulmonary hilar lymph nodes are commonly caused by the following bacterial species: Streptococcus pneumoniae, Moraxella catarrhalis, Mycoplasma pneumoniae, Klebsiella pneumoniae, Haemophilus influenza, Chlamydophila pneumoniae, Bordetella pertussis or Mycobacterium tuberculosis; or viral pathogens: influenza, adenovirus, rhinovirus, coronavirus, parainfluenza, respiratory syncytial virus, human metapneumovirus, or coxsackievirus.

Infections of the intra-abdominal lymph nodes are commonly caused by the following bacterial species: Yersinia enterocolitica, Yersinia pseudotuberculosis, Salmonella spp., Streptococcus pyogenes, Escherichia coli, Staphylococcus aureus, or Mycobacterium tuberculosis; or viral pathogens: measles, rubella, Epstein-Barr, cytomegalovirus, varicella-zoster, adenovirus, influenza, or coxsackieviruses.

Infections of the lymph nodes of the leg/inguinal region are commonly caused by the following bacterial species: Staphylococcus aureus, or Streptococcus pyogenes; or viral pathogens: measles, rubella, Epstein-Barr, cytomegalovirus, or herpes simplex.

Infections of the blood (i.e., septicemia) are commonly caused by the following bacterial species: Staphylococcus aureus, Streptococcus pyogenes, coagulase-negative staphylococci, Enterococcus spp., Escherichia coli, Klebsiella spp., Enterobacter spp., Proteus spp., Pseudomonas aeruginosa, Bacteroides fragilis, Streptococcus pneumoniae, or group B streptococci; or viral pathogens: rubeola, rubella, varicella-zoster, echoviruses, coxsackieviruses, adenovirus, Epstein-Barr, herpes simplex, or cytomegalovirus.

Infections of the bone are commonly caused by the following bacterial species: Staphylococcus aureus, coagulase-negative staphylococci, Streptococcus pyogenes, Streptococcus pneumoniae, Streptococcus agalactiae, other streptococci spp., Escherichia coli, Pseudomonas spp., Enterobacter spp., Proteus spp., or Serratia spp.; or viral pathogens: parvovirus B19, rubella, or hepatitis B.

Infections of the joint are commonly caused by the following bacterial species: Staphylococcus aureus, coagulase-negative staphylococci, Streptococcus pyogenes, Streptococcus pneumoniae, Streptococcus agalactiae, other streptococci spp., Escherichia coli, Pseudomonas spp., Enterobacter spp., Proteus spp., Serratia spp., Neisseria gonorrhea, salmonella species, Mycobacterim tuberculosis, Hemophilus influenza; or viral pathogens: parvovirus B19, rubella, hepatitis B; or fungal pathogen: Scedosporium prolificans

Infections of the meninges are commonly caused by the following bacterial species: Haemophilus influenzae, Neisseria meningitidis, Streptococcus pneumoniae, Streptococcus agalactiae, or Listeria monocytogenes; or viral pathogens: echoviruses, coxsackieviruses, other enteroviruses, or mumps.

Infections of the brain are commonly caused by the following bacterial species: Streptococcus spp. (including S. anginosus, S. constellatus, S. intermedius), Staphylococcus aureus, Bacteroides spp., Prevotella spp., Proteus spp., Escherichia coli, Klebsiella spp., Pseudomonas spp., Enterobacter spp., or Borrelia burgdorferi; or viral pathogens: coxsackieviruses, echoviruses, poliovirus, other enteroviruses, mumps, herpes simplex, varicella-zoster, flaviviruses, or bunyaviruses.

Infections of the spinal cord are commonly caused by the following bacterial species: Haemophilus influenzae, Neisseria meningitidis, Streptococcus pneumoniae, Streptococcus agalactiae, Listeria monocytogenes, or Borrelia burgdorferi; or viral pathogens: coxsackieviruses, echoviruses, poliovirus, other enteroviruses, mumps, herpes simplex, varicella-zoster, flaviviruses, or bunyaviruses.

Infections of the eye/orbit are commonly caused by the following bacterial species: Staphylococcus aureus, Streptococcus pyogenes, Streptococcus pneumoniae, Streptococcus milleri, Escherichia coli, Bacillus cereus, Chlamydia trachomatis, Haemophilus influenza, Pseudomonas spp., Klebsiella spp., or Treponema pallidum, or viral pathogens: adenoviruses, herpes simplex, varicella-zoster, or cytomegalovirus.

Infections of the salivary glands are commonly caused by the following bacterial species: Staphylococcus aureus, viridans streptococci (e.g., Streptococcus salivarius, Streptococcus sanguis, Streptococcus mutans), Peptostreptococcus spp., or Bacteroides spp., or other oral anaerobes; or viral pathogens: mumps, influenza, enteroviruses, or rabies.

Infections of the mouth are commonly caused by the following bacterial species: Prevotella melaninogenicus, anaerobic streptococci, viridans streptococci, Actinomyces spp., Peptostreptococcus spp., or Bacteroides spp., or other oral anaerobes; or viral pathogens: herpes simplex, coxsackieviruses, or Epstein-Barr.

Infections of the tonsils are commonly caused by the following bacterial species: Streptococcus pyogenes, or Group C or G B-hemolytic streptococci; or viral pathogens: rhinoviruses, influenza, coronavirus, adenovirus, parainfluenza, respiratory syncytial virus, or herpes simplex.

Infections of the sinuses are commonly caused by the following bacterial species: Streptococcus pneumoniae, Haemophilus influenza, Moraxella catarrhalis, a-streptococci, anaerobic bacteria (e.g., Prevotella spp.), or Staphylococcus aureus; or viral pathogens: rhinoviruses, influenza, adenovirus, or parainfluenza.

Infections of the nasopharynx are commonly caused by the following bacterial species: Streptococcus pyogenes, or Group C or G B-hemolytic streptococci; or viral pathogens: rhinoviruses, influenza, coronavirus, adenovirus, parainfluenza, respiratory syncytial virus, or herpes simplex.

Infections of the thyroid are commonly caused by the following bacterial species: Staphylococcus aureus, Streptococcus pyogenes, or Streptococcus pneumoniae; or viral pathogens: mumps, or influenza.

Infections of the larynx are commonly caused by the following bacterial species: Mycoplasma pneumoniae, Chlamydophila pneumoniae, or Streptococcus pyogenes; or viral pathogens: rhinovirus, influenza, parainfluenza, adenovirus, corona virus, or human metapneumovirus.

Infections of the trachea are commonly caused by the following bacterial species: Mycoplasma pneumoniae; or viral pathogens: parainfluenza, influenza, respiratory syncytial virus, or adenovirus.

Infections of the bronchi are commonly caused by the following bacterial species: Mycoplasma pneumoniae, Chlamydophila pneumoniae, Bordetella pertussis, Streptococcus pneumoniae, or Haemophilus influenzae; or viral pathogens: influenza, adenovirus, rhinovirus, coronavirus, parainfluenza, respiratory syncytial virus, human metapneumovirus, or coxsackievirus.

Infections of the lung are commonly caused by the following bacterial species: Streptococcus pneumoniae, Moraxella catarrhalis, Mycoplasma pneumoniae, Klebsiella pneumoniae, or Haemophilus influenza; or viral pathogens: influenza, adenovirus, respiratory syncytial virus, or parainfluenza.

Infections of the pleura are commonly caused by the following bacterial species: Staphylococcus aureus, Streptococcus pyogenes, Streptococcus pneumoniae, Haemophilus influenzae, Bacteroides fragilis, Prevotella spp., Fusobacterium nucleatum, peptostreptococcus spp., or Mycobacterium tuberculosis; or viral pathogens: influenza, adenovirus, respiratory syncytial virus, or parainfluenza.

Infections of the mediastinum are commonly caused by the following bacterial species: viridans streptococci, Peptococcus spp., Peptostreptococcus spp., Bacteroides spp., Fusobacterium spp., or Mycobacterium tuberculosis; or viral pathogens: measles, rubella, Epstein-Barr, or cytomegalovirus.

Infections of the heart are commonly caused by the following bacterial species: Streptococcus spp. (including S. mitior, S. bovis, S. sanguis, S. mutans, S. anginosus), Enterococcus spp., Staphylococcus spp., Corynebacterium diptheriae, Clostridium perfringens, Neisseria meningitidis, or Salmonella spp.; or viral pathogens: enteroviruses, coxsackieviruses, echoviruses, poliovirus, adenovirus, mumps, rubeola, or influenza.

Infections of the esophagus are commonly caused by the following bacterial species: Actinomyces spp., Mycobacterium avium, Mycobacterium tuberculosis, or Streptococcus spp.; or viral pathogens: cytomegalovirus, herpes simplex, or varicella-zoster.

Infections of the stomach are commonly caused by the following bacterial species: Streptococcus pyogenes or Helicobacter pylori; or viral pathogens: cytomegalovirus, herpes simplex, Epstein-Barr, rotaviruses, noroviruses, or adenoviruses.

Infections of the small bowel are commonly caused by the following bacterial species: Escherichia coli, Clostridium difficile, Bacteroides fragilis, Bacteroides vulgatus, Bacteroides thetaiotaomicron, Clostridium perfringens, Salmonella enteriditis, Yersinia enterocolitica, or Shigella flexneri; or viral pathogens: adenoviruses, astroviruses, caliciviruses, noroviruses, rotaviruses, or cytomegalovirus.

Infections of the colon/rectum are commonly caused by the following bacterial species: Escherichia coli, Clostridium difficile, Bacteroides fragilis, Bacteroides vulgatus, Bacteroides thetaiotaomicron, Clostridium perfringens, Salmonella enteriditis, Yersinia enterocolitica, or Shigella flexneri; or viral pathogens: adenoviruses, astroviruses, caliciviruses, noroviruses, rotaviruses, or cytomegalovirus.

Infections of the anus are commonly caused by the following bacterial species: Streptococcus pyogenes, Bacteroides spp., Fusobacterium spp., anaerobic streptococci, Clostridium spp., Escherichia coli, Enterobacter spp., Pseudomonas aeruginosa, or Treponema pallidum, or viral pathogens: herpes simplex.

Infections of the perineum are commonly caused by the following bacterial species: Escherichia coli, Klebsiella spp., Enterococcus spp., Bacteroides spp., Fusobacterium spp., Clostridium spp., Pseudomonas aeruginosa, anaerobic streptococci, Clostridium spp., or Enterobacter spp.; or viral pathogens: herpes simplex.

Infections of the liver are commonly caused by the following bacterial species: Escherichia coli, Klebsiella spp., Streptococcus (anginosus group), Enterococcus, spp. other viridans streptococci, or Bacteroides spp.; or viral pathogens: hepatitis A, Epstein-Barr, herpes simplex, mumps, rubella, rubeola, varicella-zoster, coxsackieviruses, or adenovirus.

Infections of the gallbladder are commonly caused by the following bacterial species: Escherichia coli, Klebsiella spp., Enterobacter spp., enterococci, Bacteroides spp., Fusobacterium spp., Clostridium spp., Salmonella enteriditis, Yersinia enterocolitica, or Shigella flexneri.

Infections of the biliary tract are commonly caused by the following bacterial species: Escherichia coli, Klebsiella spp., Enterobacter spp., enterococci, Bacteroides spp., Fusobacterium spp., Clostridium spp., Salmonella enteriditis, Yersinia enterocolitica, or Shigella flexneri; or viral pathogens: hepatitis A, Epstein-Barr, herpes simplex, mumps, rubella, rubeola, varicella-zoster, cocsackieviruses, or adenovirus.

Infections of the pancreas are commonly caused by the following bacterial species: Escherichia coli, Klebsiella spp., Enterococcus spp., Pseudomonas spp., Staphylococcal spp., Mycoplasma spp., Salmonella typhi, Leptospirosis spp., or Legionella spp.; or viral pathogens: mumps, coxsackievirus, hepatitis B, cytomegalovirus, herpes simplex 2, or varicella-zoster.

Infections of the spleen are commonly caused by the following bacterial species: Streptococcus spp., Staphylococcus spp., Salmonella spp., Pseudomonas spp., Escherichia coli, or Enterococcus spp.; or viral pathogens: Epstein-Barr, cytomegalovirus, adenovirus, measles, rubella, coxsackieviruses, or varicella-zoster.

Infections of the adrenal gland are commonly caused by the following bacterial species: Streptococcus spp., Staphylococcus spp., Salmonella spp., Pseudomonas spp., Escherichia coli, or Enterococcus spp.; or viral pathogens: varicella-zoster.

Infections of the kidney are commonly caused by the following bacterial species: Escherichia coli, Proteus mirabilis, Proteus vulgatus, Providentia spp., Morganella spp., Enterococcus faecalis, or Pseudomonas aeruginosa; or viral pathogens: BK virus, or mumps.

Infections of the ureter are commonly caused by the following bacterial species: Escherichia coli, Proteus mirabilis, Proteus vulgatus, Providentia spp., Morganella spp., or Enterococcus spp.

Infections of the bladder are commonly caused by the following bacterial species: Escherichia coli, Proteus mirabilis, Proteus vulgatus, Providentia spp., Morganella spp., Enterococcus faecalis, or Corynebacterium jekeum, or viral pathogens: adenovirus, or cytomegalovirus.

Infections of the peritoneum are commonly caused by the following bacterial species: Staphylococcus aureus, Streptococcus pyogenes, Streptococcus pneumoniae, Escherichia coli, Klebsiella spp., Proteus spp., enterococci, Bacteroides fragilis, Prevotella melaninogenica, Peptococcus spp., Peptostreptococcus spp., Fusobacterium spp., or Clostridium spp.

Infections of the retroperitoneal area are commonly caused by the following bacterial species: Escherichia coli, or Staphylococcus aureus.

Infections of the prostate are commonly caused by the following bacterial species: Escherichia coli, Klebsiella spp., Enterobacter spp., Proteus mirabilis, enterococci spp., Pseudomonas spp., Corynebacterium spp., or Neisseria gonorrhoeae; or viral pathogens: herpes simplex.

Infections of the testicle are commonly caused by the following bacterial species: Escherichia coli, Klebsiella pneumoniae, Pseudomonas aeruginosa, Staphylococcus spp., Streptococcus spp., or Salmonella enteriditis; or viral pathogens: mumps, coxsackievirus, or lymphocytic choriomeningitis virus.

Infections of the penis are commonly caused by the following bacterial species: Staphylococcus aureus, Streptococcus pyogenes, Neisseria gonorrhoeae, or Treponema pallidum, or viral pathogens: herpes simplex.

Infections of the ovary/adnexae are commonly caused by the following bacterial species: Neisseria gonorrhoeae, Chlamydia trachomatis, Gardenerella vaginalis, Prevotella spp., Bacteroides spp., Peptococcus spp. Streptococcus spp., or Escherichia coli.

Infections of the uterus are commonly caused by the following bacterial species: Neisseria gonorrhoeae, Chlamydia trachomatis, Gardenerella vaginalis, Prevotella spp., Bacteroides spp., Peptococcus spp., Streptococcus spp., or Escherichia coli.

Infections of the cervix are commonly caused by the following bacterial species: Neisseria gonorrhoeae, Chlamydia trachomatis, or Treponema pallidum, or viral pathogens: herpes simplex.

Infections of the vagina are commonly caused by the following bacterial species: Gardenerella vaginalis, Prevotella spp., Bacteroides spp., peptococci spp., Escherichia coli, Neisseria gonorrhoeae, Chlamydia Trachomatis, or Treponema pallidum, or viral pathogens: herpes simplex.

Infections of the vulva are commonly caused by the following bacterial species: Staphylococcus aureus, Streptococcus pyogenes, or Treponema pallidum, or viral pathogens: herpes simplex.

Bacterial species are classified operationally as collections of similar strains (which generally refers to groups of presumed common ancestry with identifiable physiological but usually not morphological distinctions, and which may be identified using serological techniques against bacterial surface antigens). Thus, each bacterial species (e.g., Streptococcus pneumoniae) has numerous strains (or serotypes), which may differ in their ability to cause infection or differ in their ability to cause infection in a particular organ/site. For example, although there are at least 90 serotypes of Streptococcus pneumoniae, serotypes 1, 3, 4, 7, 8, and 12 are most frequently responsible for pneumococcal disease in humans.

Certain strains of Escherichia coli, referred to as extraintestinal pathogenic E. coli (ExPEC), are more likely to cause urinary tract infection or other extraintestinal infections such as neonatal meningitis, whereas other strains, including enterotoxigenic E. coli (ETEC), enteropathogenic E. coli (EPEC), enterohemorrhagic E. coli (EHEC), Shiga toxin-producing E. coli (STEC), enteroaggregative E. coli (EAEC), enteroinvasive E. coli (EIEC) and diffuse adhering E. coli (DAEC) are more likely to cause gastrointestinal infection/diarrhea. Even among the sub-category of ExPEC strains, specific virulence factors (e.g., production of type-1 fimbriae) enable certain strains to be more capable of causing infection of the bladder, while other virulence factors (e.g., production of P fimbriae) enable other strains to be more capable of causing infection in the kidneys. In accordance with the present invention, an ExPEC strain(s) that is more likely to cause infection in the bladder may be chosen for a formulation to target immune dysregulation in the bladder cancer, whereas an ExPEC strain(s) that is more likely to cause infection in the kidney may be chosen for a formulation to target immune dysregulation in the kidney cancer. Likewise, one or more of an ETEC, EPEC, EHEC, STEC, EAEC, EIEC or DAEC strains of E. coli (i.e., strains that cause colon infection), may be chosen for a formulation to treat immune dysregulation in the colon.

Similarly, there may be numerous subtypes of specific viruses. For example, there are three types of influenza viruses, influenza A, influenza B and influenza C, which differ in epidemiology, host range and clinical characteristics. For example, influenza A is more likely to be associated with viral lung infection, whereas influenza B is more likely to be associated with myositis (i.e., muscle infection). Furthermore, each of these three types of influenza virus have numerous subtypes, which also may differ in epidemiology, host range and clinical characteristics. In accordance with the present invention, one may choose an influenza A subtype most commonly associated with lung infection to target immune dysregulation in the lung, whereas one may choose an influenza B strain most commonly associated with myositis to treat immune dysregulation in the muscle/soft tissues.

There are specific microbiota associated with some pathological tissue states, for example microbiota of specific tumours. For example, Fusobacterium and Providencia have been associated with colorectal cancer.

The compositions of the invention include immunogens of pathogenic microbial species (bacterial, viral or fungal) that are pathogenic in a specific tissue or organ, in which the immunogens are provided in the form of an artificial repertoire of mammalian PRR agonists that recapitulate a distinct portion of the PRR agonist signature of the microbial mammalian pathogen that is pathogenic in the target tissue. In select embodiments, the portion of the PRR agonist signature is distinct in the sense that it is both: different from a reference PRR agonist signature of a microbe that is not pathogenic in the target tissue; and, different than the native PRR agonist signature of the microbial mammalian pathogen. This distinct artificial repertoire of mammalian PRR agonists are formulated together in a therapeutic vehicle for combined presentation to an innate immune cell resident in the target tissue in the mammalian host.

Compositions of the invention may be provided alone or in combination with other compounds (for example, nucleic acid molecules, small molecules, peptides, or peptide analogues), in the presence of a liposome, an adjuvant, or any pharmaceutically acceptable carrier, in a form suitable for administration to mammals, for example, humans (a “therapeutic vehicle”). As used herein “pharmaceutically acceptable carrier” or “excipient” includes any and all solvents, dispersion media, coatings, antibacterial and antifungal agents, isotonic and absorption delaying agents, and the like that are physiologically compatible.

The carrier can be suitable for any appropriate form of administration, including subcutaneous, intradermal, intravenous, parenteral, intraperitoneal, intramuscular, sublingual, inhalational, intratumoural or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound (i.e., the specific bacteria, bacterial antigens, or compositions thereof of the invention), use thereof in the pharmaceutical compositions of the invention is contemplated. Supplementary active compounds can also be incorporated into the compositions.

Aspects of the invention involve the use of nanoparticle (NP) formulations. For example, virus-like particles (VLPs) are in essence empty viral particles with an intact protein hull and, in some embodiments, membrane envelopes. In general, VLPs lack genetic material. Production of VLPs may for example be by expression of viral proteins in mammalian, avian, insect, plant, yeast, or bacterial cells. Alternatively, fully synthetic VLPs may be produced. Alternative nanoparticle formulations emulsions, liposomes alginates, chitosan, and polylactide-coglycolide (PLGA) NPs. Examples of NP/TLR ligand preparations that may be adapted for use to induce immune responses are ligands for TLR2 (Pam(3)Cys), TLR9 (Poly I: C), TLR4 (3-O-desacyl-4 0-monophosphoryl lipid A (MPL)), TLR7 (9-benzyl-8-hydroxyadenine), TLR7/8 (resiquimod, R848), and TLR9 (CpG DNA).

In addition to selected co-formulations, a wide variety of adjuvants may be used to potentiate a desired immune response (see Levast et al., 2014, Vaccines, 2, 297-322).

Treatment with PRR ligands according to the invention may be combined with more traditional and existing therapies. For cancer, for example, these may include chemotherapy, radiation therapy, surgery, etc., or with a therapy that stimulates the immune system, reduces inflammation or otherwise benefits the subject, such as nutrients, vitamins and supplements. For example, vitamin A, vitamin D, vitamin E, vitamin C, vitamin B complex, selenium, zinc, co-enzyme Q10, beta carotene, fish oil, curcumin, green tea, bromelain, resveratrol, ground flaxseed, garlic, lycopene, milk thistle, melatonin, other antioxidants, cimetidine, indomethacin, or COX-2 Inhibitors (e.g., Celebrex™ [celecoxib] or Vioxx™ [rofecoxib]) may be also be administered to the subject.

Conventional pharmaceutical practice may be employed to provide suitable formulations or compositions to administer the compounds to subjects. Alternative routes of administration may be employed, for example, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, intratumoural, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets.

Methods well known in the art for making formulations are found in, for example, “Remington's Pharmaceutical Sciences” (20th edition), ed. A. Gennaro, 2000, Mack Publishing Company, Easton, PA. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic compositions, the pathogenic bacterial species are administered to an individual in an amount effective to stop or slow progression or metastasis of the cancer, or to increase survival of the subject (relative to, for example, prognoses derived from the SEER database) depending on the disorder.

Biodegradable polymeric materials may be used for the staged delivery of antigenic and immunomodulatory therapeutics, including chitosan, alginate, gelatin, poly(lactic-co-glycolic acid) (PLGA), Poly(lactic acid) (PLA), poly(c-caprolactone) (PCL), poly(p-amino esters) (PBAE) and poly(methyl methacrylate) (PMMA) (Lu et al., 2021). Similarly, bioerodible systems have been developed that are capable of delivering one or more therapeutics in a predetermined temporal sequence (Sundararaj et al., 2014). Polyanhydrides and poly(ortho esters) are examples of surface-eroding polymers employed for controlled delivery of therapeutics in this way. Similarly, cellulose acetate phthalate (CAP) and Pluronic F-127 (P), when mixed in an aprotic solvent, associate via hydrogen bonds to provide a matrix capable of mediating the release of therapeutic compositions. The CAP to Pluronic ratio may be adjusted to modulate the erosion rate and thereby the timing of therapeutic release. These systems may accordingly be provided in a staged-release matrix encasing a therapeutic, such as an attenuated or killed mammalian pathogen, for intermittent staged-release, adapted to release the mammalian pathogen from the matrix in a plurality of successive temporally separated dosing stages, for example after a delivery system is applied to a surgical wound on a surgery date, with a therapeutically effective alliquote of mammalian pathogen being released at each dosing stage during a perioperative period, for example a period that is within 1, 2, 3, 4, 5 or 6 months of the surgery date.

Pharmaceutical compositions or formulations may be packaged in a variety of ways depending upon the method used for administering the drug. For example, an article of manufacture or package may include a container having deposited therein the pharmaceutical formulation in an appropriate form. Suitable containers may for example include materials such as bottles (plastic and glass), sachets, ampoules, plastic bags, metal cylinders, and vials. The container may have a sterile access port, for example the container may be an intravenous solution bag or a vial having a stopper pierceable by a hypodermic injection needle. The package or container may also include a tamper-proof or multi-use mechanism adapted to control access to the contents of the package or the container, for example a multi dose vial adapter matched to a vial contained in the package. The container or package may include a label, for example a lable that describes the contents of the container, for example a drug label identifying the pharmaceutical composition therein and/or specifying modes or routes of administration. The label may also include appropriate warnings, for example specifying storage conditions for the container or package, or setting out contraindications or adverse effects of a mode of treatment. Articles of manufacture may accordingly take the form of a “kit” comprising pharmaceutical compositions or accessories adapted to facilitate use of pharmaceutical compositions. Kits may include a label or package insert, where the term “package insert” is used to refer to instructions customarily included in commercial packages of therapeutic products, that contain information about the indications, usage, dosage, administration, contraindications and/or warnings concerning the use of such therapeutic products. Kits may further include accessories associated with use of the pharmaceutical composition, including buffers, diluents, filters, needles, and syringes. Kits may also be adapted for the delivery of selected dosage forms of a pharmaceutical composition, for example including a number of unit dosages. Such kits can include a memory aid or mechanism, in the form of a physical or written indication of the intended timing of a treatment schedule in which the dosages are to be used.

A “companion diagnostic” may be associated with a pharmaceutical treatment or composition. Companion diagnostics are assays that facilitate the associated treatment, by providing diagnostic or prognostic information, typically in the form of a diagnostic test to determine the applicability of a treatment to a specific patient. Point-of-care companion diagnostics may for example involve providing diagnostic compositions and/or articles of manufacture in conjunction with providing a pharmaceutical formulation, for example as part of a kit. Alternatively, companion diagnostics may be separately provided, as assays to monitor the therapy of subjects or to predict the therapeutic efficacy of an intended treatment. A companion diagnostic may for example take the form of a medical device, such as an imaging tool, or a process carried out by such a device, for example for conducting assays in vitro, which provides information that is relevant for the safe and effective use of a corresponding drug or biological product. Companion diagnostics may be used with therapies disclosed herein so as to provide diagnostic or prognostic information about therapeutic efficacy or evidence of undesirable side effects or risks. The use of a companion diagnostic with a particular therapeutic may be stipulated in instructions, for example on the labeling of a diagnostic device and/or the labeling of the corresponding therapeutic product. Types of companion diagnostic tests may for example include: screening and detection, in form of tests that screen for genetic patterns, such as genetic SSI response markers; prognosis and theranostics, such as assays for biochemical SSI response markers that help to predict the future course of a disease, or indicate a patient's response to a therapy; monitoring, for example to evaluate the effectiveness and appropriate dosing of a prescribed therapy; or, recurrence, involving tests that analyze the patient's risk for a recurrence of the disease.

An “effective amount” of a composition according to the invention includes a therapeutically effective amount or a prophylactically effective amount. A “therapeutically effective amount” refers to an amount effective, at dosages and for periods of time necessary, to achieve the desired therapeutic result, such as reduction or elimination of the immune dysregulation. A therapeutically effective amount of a composition may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the composition are outweighed by the therapeutically beneficial effects. A “prophylactically effective amount” refers to an amount effective, at dosages and for periods of time necessary, to achieve the desired prophylactic result, such as amelioration of immune dysregulation. Typically, a prophylactic dose is used in subjects prior to or at an earlier stage of cancer, so that a prophylactically effective amount may be less than a therapeutically effective amount.

For any particular subject, the timing and dose of treatments may be adjusted over time (e.g., timing may be daily, every other day, weekly, monthly) according to the individual need and the professional judgment of the person administering or supervising the administration of the compositions. For example, in the context of subcutaneous or intradermal administration, the compositions may be administered every second day. An initial dose of approximately 0.05 ml may be administered subcutaneously, followed by increases from 0.01-0.02 ml every second day until an adequate skin reaction is achieved at the injection site (for example, a 1 inch to 2 inch diameter delayed reaction of visible redness at the injection site). Once this adequate immune reaction is achieved, this dosing is continued as a maintenance dose. The maintenance dose may be adjusted from time to time to achieve the desired visible skin reaction (inflammation) at the injection site. Dosing may be for a dosage duration, for example of at least 1 week, 2 weeks, 2 months, 6 months, 1, 2, 3, 4, or 5 years or longer.

Oral dosages may for example range from 4 times per day, daily or weekly. Dosing may be for a dosage duration, for example of at least 1 week, 2 weeks, 2 months, 6 months, 1, 2, 3, 4, or 5 years or longer. In some embodiments, the invention may include compositions administered sublingually or by inhalation, or administered to one or more epithelial tissues (i.e., skin by intradermal or subcutaneous injection; lung epithelium by inhalation; gastrointestinal mucosa by oral ingestion; mouth mucosa by sublingual administration) simultaneously or sequentially. Accordingly, in some embodiments the compositions of the invention are administered so as to provoke an immune response in an epithelial tissue. In some embodiments, one or more epithelial routes of administration may be combined with one or more additional routes of administration, such as intratumoural, intramuscular or intravenous administration.

In the case of immunogenic formulations, an immunogenically effective amount of a composition of the invention can be provided, alone or in combination with other compounds, for example with an immunological adjuvant. The composition may for example include compounds linked with a carrier molecule, such as bovine serum albumin or keyhole limpet hemocyanin to enhance immunogenicity. An immunogenic composition is a composition that includes materials that elicit a desired immune response. An immunogenic composition may select, activate or expand, without limitation: memory B, T cells, neutrophils, monocytes or macrophages of the immune system.

An antigenic composition comprising killed recombinant bacteria for administration by injection may be made as follows. The bacteria may be grown in suitable media, and washed with physiological salt solution. The bacteria may then be centrifuged, resuspended in saline solution, and killed with heat. The suspensions may be standardized by direct microscopic count, mixed in required amounts, and stored in appropriate containers, which may be tested for safety, shelf life, and sterility in an approved manner. In addition to the pathogenic bacterial species and/or antigens thereof, a killed bacterial vaccine suitable for administration to humans may include 0.4% phenol preservative and/or 0.9% sodium chloride. The bacterial vaccine may also include trace amounts of brain heart infusion (beef), peptones, yeast extract, agar, sheep blood, dextrose, sodium phosphate and/or other media components.

In select embodiments, medicaments may be administered at an administration site in successive doses given at a dosage interval of between one hour and one month, over a dosage duration of at least one week. Optionally, the medicament may be administered intradermally or subcutaneously. Optionally, the medicament may be administered in a dose so that each dose is effective to cause a visible localized inflammatory immune response at the administration site. Optionally, the medicament may be administered so that visible localized inflammation at the administration site occurs within 1 to 48 hours. However, a visible localized inflammatory immune response may not always be present in all circumstances despite an immune response being initiated. There are other methods by which the mounting of an immune response can be monitored. For example, the profile (and relative change in characterization) of immune cells from a subject undergoing an immune reaction can be compared with those from a subject that is not undergoing an immune reaction.

In another aspect, a method of monitoring efficacy of a treatment regime in an individual being treated for an immune dysfunction in a specific organ or tissue is provided. The method involves measuring a characteristic of an immune response in a post-treatment immune sample obtained from the specific organ or tissue after the individual has been subject to the treatment regime for a period of time.

In some embodiments, PRR agonists derived from bacteria that are members of the endogenous flora of a particular region of the GIT may be used to formulate immunogenic compositions of the invention. The rows of Table 3 list a number of bacterial species, together with the biological regions in which each species may form a part of the endogenous flora. For example, Abiotrophia spp. are typically members of the endogenous flora of the mouth.

TABLE 3
Human Bacterial Normal Flora (Endogenous
Bacterial Human Pathogens)

| | | | Duodenum/ | | |
|---|---|---|---|---|---|
| Bacterial species | Mouth | Stomach | Jejunum | Ileum | Colon |
| CFU/mL | 10\hat{ }5 | 10\hat{ }2 | 10\hat{ }5 | 10\hat{ }8 | 10\hat{ }11 |
| Abiotrophia spp | + | | | | |
| Acholeplasma | + | | | | |
| laidlawii | | | | | |
| Acidaminococcus | + | | + | + | + |
| fermentans | | | | | |
| Acinetobacter | + | | + | + | + |
| spp. | | | | | |
| Actinobacillus | + | | | | |
| spp. | | | | | |
| Actinobaculum | + | | + | + | + |
| spp. | | | | | |
| Actinomyces | + | | + | + | + |
| spp. | | | | | |
| Aeromonas spp. | | | + | + | + |
| Anaerorhabdus | | | | + | + |
| furcosus | | | | | |
| Anaerococcus | | | | + | + |
| hydrogenalis | | | | | |
| Anaerococcus | | | | + | + |
| lactolyticus | | | | | |
| Anaerococcus | | | | + | + |
| prevotii | | | | | |
| Atopobium spp. | + | | + | + | + |
| Bacillus spp. | | | | + | + |
| Bacteroides | | | | + | + |
| caccae | | | | | |
| Bacteroides | | | | + | + |
| distasonis | | | | | |
| Bacteroides | | | | + | + |
| eggerthii | | | | | |
| Bacteroides | | | | + | + |
| fragilis | | | | | |
| Bacteroides | | | | + | + |
| merdae | | | | | |
| Bacteroides | | | | + | + |
| ovatus | | | | | |
| Bacteroides | | | | + | + |
| splanchnicus | | | | | |
| Bacteroides | | | | + | + |
| thetaiotaomicron | | | | | |
| Bacteroides | | | | + | + |
| vulgatus | | | | | |
| Bifidobacterium | | | + | + | + |
| adolescentis | | | | | |
| Bifidobacterium | | | + | + | + |
| bifidum | | | | | |
| Bifidobacterium | | | + | + | + |
| breve | | | | | |
| Bifidobacterium | | | + | + | + |
| catenulatum | | | | | |
| Bifidobacterium | + | | + | + | + |
| dentium | | | | | |
| Bifidobacterium | | | + | + | + |
| longum | | | | | |
| Bilophila | + | | + | + | + |
| wadsworthia | | | | | |
| Burkholderia | | | + | + | + |
| cepacia | | | | | |
| Butyrivibrio | | | + | + | + |
| fibrisolvens | | | | | |
| Campylobacter | | | + | + | + |
| concisus | | | | | |
| Campylobacter | | | + | + | + |
| curvus | | | | | |
| Campylobacter | | | + | + | + |
| gracilis | | | | | |
| Campylobacter | | | + | + | + |
| jejuni | | | | | |
| Campylobacter | | | + | + | + |
| rectus | | | | | |
| Campylobacter | + | | + | + | + |
| showae | | | | | |
| Campylobacter | + | | | | |
| sputorum | | | | | |
| Capnocytophaga | + | | | | |
| granulosum | | | | | |
| Capnocytophaga | + | | | | |
| gingivalis | | | | | |
| Campylobacter | + | | | | |
| haemolytica | | | | | |
| Capnocytophaga | + | | + | + | + |
| ochracea | | | | | |
| Capnocytophaga | + | | | | |
| sputigena | | | | | |
| Cardiobacterium | + | | | | |
| hominis | | | | | |
| Cedecea spp | | | | | + |
| Centipeda | + | | | | |
| periodontii | | | | | |
| Citrobacter | | | + | + | + |
| freundii | | | | | |
| Citrobacter | | | + | + | + |
| koseri | | | | | |
| Clostridium spp. | | | + | + | + |
| Corynebacterium | + | | | | |
| accolens | | | | | |
| Corynebacterium | + | | | | |
| afermentans | | | | | |
| Desulfomonas | | | + | + | + |
| pigra | | | | | |
| Dysgonomonas | | | + | + | + |
| spp. | | | | | |
| Eikenella | + | | + | + | + |
| corrodens | | | | | |
| Enterobacter | | | + | + | + |
| aerogenes | | | | | |
| Enterobacter | | | + | + | + |
| cloacae | | | | | |
| Enterobacter | | | + | + | + |
| gergoviae | | | | | |
| Enterobacter | | | + | + | + |
| sakazakii | | | | | |
| Enterobacter | | | + | + | + |
| taylorae | | | | | |
| Enterococcus | | | + | + | + |
| spp. | | | | | |
| Escherichia coli | | | + | + | + |
| Escherichia | | | + | + | + |
| fergusonii | | | | | |
| Escherichia | | | + | + | + |
| hermannii | | | | | |
| Escherichia | | | + | + | + |
| vulneris | | | | | |
| Eubacterium | + | | + | + | + |
| spp. | | | | | |
| Ewingella | + | | | | |
| americana | | | | | |
| Finegoldia | | | + | + | + |
| magnus | | | | | |
| Fusobacterium | + | | | | |
| alocis | | | | | |
| Fusobacterium | | | + | + | + |
| gonidiaformans | | | | | |
| Fusobacterium | | | + | + | + |
| mortiferum | | | | | |
| Fusobacterium | | | + | + | + |
| naviforme | | | | | |
| Fusobacterium | + | | + | + | + |
| necrophorum | | | | | |
| Fusobacterium | + | | | | + |
| nucleatum | | | | | |
| Fusobacterium | + | | | | |
| sulci | | | | | |
| Fusobacterium | | | + | + | + |
| russii | | | | | |
| Fusobacterium | | | + | + | + |
| varium | | | | | |
| Gardnerella | | | + | + | + |
| vaginalis | | | | | |
| Gemella | + | | | | |
| haemolysans | | | | | |
| Gemella | + | | + | + | + |
| morbillorum | | | | | |
| Globicatella spp. | + | | | | + |
| Granulicatella | + | | | | |
| spp. | | | | | |
| Haemophilus | + | | | | |
| spp. | | | | | |
| Hafnia alvei | | | + | + | + |
| Helcococcus | | | | | |
| kunzii | | | | | |
| Helicobacter spp. | | | + | + | + |
| Kingella spp. | + | | | | |
| Klebsiella spp. | + | | + | + | + |
| Lactobacillus | + | + | + | + | + |
| acidophilus | | | | | |
| Lactobacillus | + | | | | |
| breve | | | | | |
| Lactobacillus | + | | | | |
| casei | | | | | |
| Lactobacillus | + | + | + | + | + |
| fermentum | | | | | |
| Lactobacillus | | + | + | + | + |
| reuteri | | | | | |
| Lactobacillus | + | + | + | + | + |
| salivarius | | | | | |
| Leclercia | | | + | + | + |
| adecarboxylata | | | | | |
| Leminorella spp. | | | + | + | + |
| Leptotrichia | + | | | | |
| buccalis | | | | | |
| Megasphaera | | | + | + | + |
| elsdenii | | | | | |
| Micrococcus | + | | | | |
| luteus | | | | | |
| Micrococcus | + | | | | |
| lylae | | | | | |
| Micromonas | + | | | | |
| micros | | | | | |
| Mitsuokella | | | + | + | + |
| multiacidus | | | | | |
| Mobiluncus | | | + | + | + |
| curisii | | | | | |
| Mobiluncus | | | + | + | + |
| mulieris | | | | | |
| Moellerella | | | + | + | + |
| wisconsensis | | | | | |
| Moraxella | + | | | | |
| catarrhalis | | | | | |
| other Moraxella | + | | | | |
| spp. | | | | | |
| Morganella | | | + | + | + |
| morganii | | | | | |
| Mycoplasma | + | | | | |
| buccale | | | | | |
| Mycoplasma | + | | | | |
| fermentans | | | | | |
| Mycoplasma | + | | | | |
| hominis | | | | | |
| Mycoplasma | + | | | | |
| lipophilum | | | | | |
| Mycoplasma | + | | | | |
| orale | | | | | |
| Mycoplasma | + | | | | |
| pneumoniae | | | | | |
| Mycoplasma | + | | | | |
| salivarium | | | | | |
| Pantoea | | | + | + | + |
| agglomerans | | | | | |
| Pasteurella | + | | | | |
| multocida | | | | | |
| Pediococcus | + | | | | + |
| spp. | | | | | |
| Peptoniphilus | | | + | + | + |
| asaccharolyticus | | | | | |
| Peptostreptococcus | + | | + | + | + |
| anaerobus | | | | | |
| Peptostreptococcus | | | + | + | + |
| productus | | | | | |
| Porphyromonas | + | | + | + | + |
| asaccharolytica | | | | | |
| Porphyromonas | + | | + | | |
| catoniae | | | | | |
| Porphyromonas | + | | + | | |
| endodontalis | | | | | |
| Porphyromonas | + | | + | | |
| gingivalis | | | | | |
| Prevotella | + | | + | | |
| buccae | | | | | |
| Prevotella | + | | + | | |
| buccalis | | | | | |
| Prevotella | + | | + | | |
| corporis | | | | | |
| Prevotella | + | | + | | |
| dentalis | | | | | |
| Prevotella | + | | + | | |
| denticola | | | | | |
| Prevotella | + | | + | | |
| enoeca | | | | | |
| Prevotella | + | | + | | |
| heparinolytica | | | | | |
| Prevotella | + | | + | | |
| intermedia | | | | | |
| Prevotella | + | | + | | |
| loescheii | | | | | |
| Prevotella | + | | + | | |
| melaninogenica | | | | | |
| Prevotella | + | | + | | |
| nigrescens | | | | | |
| Prevotella oralis | + | | + | | |
| Prevotella oris | + | | + | | |
| Prevotella | + | | + | | |
| oulorum | | | | | |
| Prevotella | + | | + | | |
| tannerae | | | | | |
| Prevotella | + | | + | | |
| veroralis | | | | | |
| Prevotella | + | | + | | |
| zoogleoformans | | | | | |
| Propionibacterium | + | | | | |
| propionicum | | | | | |
| Proteus mirabilis | | | | + | + |
| Proteus penneri | | | | + | + |
| Proteus vulgaris | | | | + | + |
| Providencia | | | | + | + |
| rettgeri | | | | | |
| Providencia | | | + | + | + |
| stuartii | | | | | |
| Pseudomonas | | | + | + | + |
| aeruginosa | | | | | |
| Retortamonas | | | + | + | + |
| intestinalis | | | | | |
| Rothia | + | | | | |
| dentocariosa | | | | | |
| Rothia | + | | | | |
| mucilaginosa | | | | | |
| Ruminococcus | | | + | + | + |
| productus | | | | | |
| Selenomonas | + | | | | |
| spp. | | | | | |
| Serratia | | | | + | + |
| liquefaciens | | | | | |
| Serratia | | | | + | + |
| marcescens | | | | | |
| Serratia odorifera | | | | + | + |
| Staphylococcus | + | | | | |
| aureus | | | | | |
| Staphylococcus | + | | | | |
| epidermidis | | | | | |
| Streptococcus | | | + | + | + |
| agalactiae | | | | | |
| Streptococcus | + | | + | + | + |
| anginosus | | | | | |
| Streptococcus | | | + | + | + |
| bovis | | | | | |
| Streptococcus | + | | + | + | + |
| constellatus | | | | | |
| Streptococcus | + | | | | |
| criceti | | | | | |
| Streptococcus | + | | | | |
| crista | | | | | |
| Streptococcus | + | | | | |
| equisimilis | | | | | |
| Streptococcus | + | | | | |
| gordonii | | | | | |
| Streptococcus | + | | | + | + |
| intermedius | | | | | |
| Streptococcus | + | + | | | |
| mitis | | | | | |
| Streptococcus | + | | | | |
| mutans | | | | | |
| Streptococcus | + | | | | |
| oralis | | | | | |
| Streptococcus | + | | | | |
| parasanguis | | | | | |
| Streptococcus | + | + | | | |
| pyogenes | | | | | |
| Streptococcus | + | + | | | |
| salivarius | | | | | |
| Streptococcus | + | + | | | |
| sanguis | | | | | |
| Streptococcus | + | | | | |
| sobrinus | | | | | |
| Streptococcus | + | | | | |
| vestibularis | | | | | |
| Group C + G | + | | | | + |
| Streptococci | | | | | |
| Succinivibrio | | | + | + | + |
| dextrinosolvens | | | | | |
| Sutterella spp. | + | | | + | + |
| Suttonella | + | | | | |
| indologenes | | | | | |
| Tissierella | | | + | + | + |
| praeacuta | | | | | |
| Treponema | + | | | | |
| denticola | | | | | |
| Treponema | + | | | | |
| maltophilum | | | | | |
| Treponema | + | | | | |
| socranskii | | | | | |
| Treponema | + | | | | |
| vincentii | | | | | |
| Ureaplasma | + | | | | |
| urealyticum | | | | | |
| Veillonella spp. | + | | + | + | + |

Endogenous microbial flora, such as bacteria, have access to tissues for pathogenesis either through contiguous spread or bacteremic spread. Under favorable conditions, endogenous organisms can become pathogenic and invade locally and spread by contiguous spread to adjacent tissues and organs. Endogenous bacterial flora of the skin, mouth and colon are species that are understood to also be amenable to bacteremic spread. Bacteria that are members of a particular endogenous flora domain may therefore cause infection in tissues or organs to which these bacteria may spread. Accordingly, one aspect of the invention involves the use of PRR agonists derived from endogenous microbial pathogens to treat an immune dysregulation having symptoms localized to a region of the GIT in which the endogenous bacteria may spread to cause infection. The columns of Table 2 list domains for endogenous flora. The rows of Table 4 list regions of the GIT within which immune dysregulation may be symptomatic or etiologically located. Accordingly, one aspect of the invention involves the use of PRR agonists derived from endogenous microbial pathogens to formulate immunogenic compositions for treating an immune dysregulation symptomatic or etiologically located in the region of the GIT to which the pathogen may spread to cause an infection. Accordingly, in alternative embodiments, an immune dysregulation that is symptomatic in the region listed in the first column of Table 2 may be treated with immunogenic compositions comprising an artificial repertoire of mammalian PRR agonists that recapitulates a distinct portion of a PRR agonist signature of a microbial mammalian pathogen that is a member of the endogenous flora of one or more of the endogenous flora domains listed in the first row of Table 2 and indicated with an X or a check mark in the appropriate row.

TABLE 4
Tissue/Organ Pathogenicity of Endogenous Flora

| | organ site | | | | |
|---|---|---|---|---|---|
| | | | Duodenum/ | | |
| Tissue | Mouth | Stomach | Jejunum | Ileum | Colon |
| Oral | x | | | | |
| Tonsil | x | | | | |
| Nasopharynx/ | x | | | | |
| Sinus | | | | | |
| Esophagus | | x | | | |
| Stomach | | x | | | |
| Small bowel | | | x | x | |
| Colon/ | | | | | x |
| Rectum | | | | | |
| Anus | | | | | x |

In accordance with the combined information in Tables 1 and 2, an immune dysregulation manifest in a particular region of the GIT set out in column 1 of Table 2 may be treated with antigenic compositions comprising an artificial repertoire of mammalian PRR agonists that recapitulates a distinct portion of a PRR agonist signature of a microbial mammalian pathogen that is one of the corresponding bacterial species of Table 1, so that the column headings in Table 2 are in effect replaced with the bacterial species of Table 1.

In some embodiments, PRR agonists may be derived from exogenous bacterial pathogens. For example, PRR agonists derived from the organisms listed in Table 5 may be used in an artificial repertoire of PRR agonists to treat an immune dysregulation that is symptomatic in the region of the GIT listed with the relevant organism in Table 5. In some embodiments, PRR agonists derived from both endogenous and exogenous microbial species may be used in combination.

TABLE 5
Exogenous Bacterial Human Pathogens, and
their Sites of Infection in the GIT.

| Bacterial Species | Region of the GIT |
|---|---|
| Aerobacter spp. | small bowel, colon, |
| Bacillus anthracis | oral, small bowel, colon, hematological |
| Bacillus cereus | colon, |
| other Bacillus spp. | colon, stomach, small bowel |
| Brucella spp. | small bowel, colon |
| Campylobacter coli | small bowel, colon |
| Campylobacter | colon |
| jejuni | |
| Campylobacter | small bowel, colon |
| sputorum | |
| Clostridium | small bowel, colon, stomach |
| bifermentans | |
| Clostridium | colon, small bowel |
| botulinum | |
| Clostridium difficile | colon |
| Clostridium indolis | small bowel, colon, stomach, |
| Clostridium | small bowel, colon, stomach |
| mangenolii | |
| Clostridium | small bowel, colon, stomach |
| perfringens | |
| Clostridium sordellii | small bowel, colon, stomach |
| Clostridium | small bowel, colon, stomach |
| sporogenes | |
| Clostridium | small bowel, colon, stomach |
| subterminale | |
| Edwarsiella tarda | small bowel, colon |
| Francisella | small bowel |
| tularensis | |
| Helicobacter pylori | stomach |
| Leptospirosis spp. | oral |
| Listeria | small bowel, colon |
| monocytogenes | |
| Mycobacterium | colon, small bowel |
| bovis | |
| Mycobacterium | small bowel, colon |
| tuberculosis | |
| Pediococcus spp. | colon |
| Plesiomonas | small bowel, colon |
| shigelloides | |
| Rickettsia | small bowel |
| rickettsiae | |
| Salmonella spp. | stomach, small bowel, colon |
| Shigella boydii | colon |
| Shigella | colon |
| dysenteriae | |
| Shigella flexneri | colon |
| Shigella sonnei | colon |
| other Spirillum spp. | colon |
| Streptococcus | small bowel |
| zooepidemicus | |
| Treponema | oral, anus |
| pallidum | |
| Tropheryma | small bowel, colon |
| whipplei | |
| Vibrio cholerae | colon, small bowel |
| Vibrio fluvialis | small bowel, colon |
| Vibrio furnissii | small bowel, colon |
| Vibrio hollisae | small bowel, colon |
| Vibrio | colon, small bowel |
| parahaemolyticus | |
| Yersinia | small bowel, colon |
| enterocolitica | |
| Yersinia | small bowel, colon |
| pseudotuberculosis | |

In some embodiments, PRR agonists for use in the invention may be derived from viral pathogens. Table 6 provides an exemplary list of viral pathogens together with the tissue and organ sites for which each viral species is reportedly a pathogen. Accordingly, one aspect of the invention involves utilizing immunogenic compositions of PRR agonists derived from the named viruses to treat an immune dysregulation that is symptomatic in the region of the GIT that is identified adjacent to the name of the virus in Table 6.

TABLE 6
Viral Human Pathogens and Their Sites of Infection

| Virus | Region of the GIT |
|---|---|
| Herpes Simplex | rectum, anus |
| virus (1 and 2) | |
| Cytomegalovirus | small bowel, colon/rectum |
| Epstein-Barr virus | oral |
| Adenovirus | oral, small bowel, colon |
| Human | anus, oral |
| papillomavirus | |
| Orthoreoviruses | small bowel, colon, oral |
| Coltiviruses | oral |
| Rotaviruses | small bowel, colon |
| Alphaviruses | small bowel, colon, |
| Coronaviruses | oral, small bowel, colon |
| Toroviruses | small bowel, colon |
| Parainfluenza | oral |
| viruses | |
| Respiratory syncytial | oral |
| virus | |
| Human | oral, small bowel, colon |
| metapneumovirus | |
| Vesicular stomatitis | oral, small bowel, colon |
| virus | |
| Rabies virus | oral |
| Influenza virus | oral |
| Hantaviruses | oral |
| Machupo virus | small bowel, colon |
| Junin virus | small bowel, colon |
| Poliovirus | small bowel, colon |
| Coxsackieviruses | small bowel, colon |
| Echoviruses | oral, small bowel, colon |
| Hepatitis A virus | small bowel, colon |
| Rhinoviruses | oral |
| Noroviruses and | small bowel, colon |
| other Caliciviruses | |
| Astroviruses | small bowel, colon |
| Picobirnaviruses | small bowel, colon |
| Hepatitis E virus | small bowel, colon |

In some embodiments, the pathogen from which PRR agonists are derived for use in immunogenic compositions of the invention may be one that is a common cause of acute infection in the region of the GIT in which the immune dysregulation to be treated is symptomatic. Table 7 identifies bacterial and viral pathogens of this kind, together with the region of the GIT in which they commonly cause infection. Accordingly, in selected embodiments, an immune dysregulation that is symptomatic in a region of the GIT identified in the first column of Table 7 may be treated with an immunogenic composition that comprises an artificial repertoire of mammalian PRR agonists that recapitulates a distinct portion of the PRR agonist signature of a pathogenic organism listed in the second column of Table 7.

TABLE 7
Common causes of acute infection (bacteria and
viruses) for selected regions of the GIT

| Selected regions | |
|---|---|
| of the GIT | Common Bacterial or Viral Pathogens |
| Oral | Prevotella melaninogenicus, anaerobic streptococci, |
| | viridans streptococci, Actinomyces spp., |
| | Peptostreptococcus spp., Bacteroides spp., and other oral |
| | anaerobes |
| | herpes simplex, coxsackieviruses, Epstein-Barr |
| Stomach | Streptococcus pyogenes, Helicobacter pylori |
| | cytomegalovirus, herpes simplex, Epstein-Barr, rotaviruses, |
| | noroviruses, adenoviruses |
| Small bowel | Escherichia coli, Clostridium difficile, Bacteroides fragilis, |
| | Bacteroides vulgatus, Bacteroides thetaiotaomicron, |
| | Clostridium perfringens, Salmonella enteriditis, |
| | Yersinia enterocolitica, Shigella flexneri |
| | adenoviruses, astroviruses, caliciviruses, noroviruses, |
| | rotaviruses, cytomegalovirus |
| Colon/Rectum | Escherichia coli, Clostridium difficile, Bacteroides fragilis, |
| | Bacteroides vulgatus, Bacteroides thetaiotaomicron, |
| | Clostridium perfringens, Salmonella enteriditis, |
| | Yersinia enterocolitica, Shigella flexneri |
| | adenoviruses, astroviruses, caliciviruses, noroviruses, |
| | rotaviruses, cytomegalovirus |
| Anus | Streptococcus pyogenes, Bacteroides spp., Fusobacterium |
| | spp., anaerobic streptococci, Clostridium spp., E. coli, |
| | Enterobacter spp., Pseudomonas aeruginosa, Treponema pallidum |
| | herpes simplex |

Humans are hosts to a wide range of gastrointestinal parasites, including various protozoa and helminths, which for purposes of the present invention constitute pathogens of the GIT (Schafer, T. W., Skopic, A. Parasites of the small intestine. Curr Gastroenterol Reports 2006; 8:312-20; Jernigan, J., Guerrant, R. L., Pearson, R. D. Parasitic infections of the small intestine. Gut 1994; 35:289-93; Sleisenger & Fordtran's Gastrointestinal and liver disease. 8th ed. 2006; Garcia, L. S. Diagnostic medical parasitology. 5th ed. 2007). Compositions of the invention may accordingly include PRR agonists of various protozoa, including for example: Giardia lamblia, Cryptosporidium parvum, Cryptosporidium hominus, Isospora belli, Sarcocystis species, Coccidian like bodies (Cyclospora species), Enterocytozoon bieneusi, Entamoeba histolytica, Entamoeba dispar, Entamoeba coli, Entamoeba hartmanni, Endolimax nana, Iodamoeba batschlii, Dientameoba fragilis, Blastocystis hominus, Cyclospora cayetanensis, Microsporidia, Trypanosoma cruzi, Chilomastix mesnili, Pentatrichomonas hominis, Balantidium coli. Similarly, compositions of the invention may include antigenic components of various helminths, including for example: Cestodes (tapeworms), Taenia saginata, Taenia solium, Diphyllobothrium species, Hymenolepis nana, Hymenolepis diminuta, Dipylidium caninum, Nematodes (round worms), Ascaris lumbricoides, Strongyloides stercoralis, Necator americanus, Ancylostoma duodenale, Ancylostoma caninum, Tichuris trichiura, Capillaria philippinensis, Trichostrongylus species, Trichinella species, Necator americanus, Anisakis and related species, Angiostrongylus costaricensis, Enterobius vermicularis, Trematodes (flukes), Fasciolopsis buski, Heterophyes speicies, Echinostoma species, Clonorchis sinensis, Opisthorchis species, Fasciola species, Metagonimus yokogawi, Schistosoma mansoni, Schistosoma japonicum, Schistosoma mekongi, Schistosoma intercalatum, Echinostoma species and Paragonimus species.

In accordance with the foregoing, in various aspects, the invention may involve the treatment of an immune dysregulation with formulations of an artificial repertoire of mammalian PRR agonists that recapitulates a distinct portion of a PRR agonist signature of a microbial pathogen that is an: Acidaminococcus fermentans; Acinetobacter spp.; Actinobaculum spp.; Actinomyces spp.; Aeromonas spp.; Anaerorhabdus furcosus; Anaerococcus hydrogenalis; Anaerococcus lactolyticus; Anaerococcus prevotii; Atopobium spp.; Bacillus spp.; Bacteroides caccae; Bacteroides distasonis; Bacteroides eggerthii; Bacteroides fragilis; Bacteroides merdae; Bacteroides ovatus; Bacteroides splanchnicus; Bacteroides thetaiotaomicron; Bacteroides vulgatus; Bifidobacterium adolescentis; Bifidobacterium bifidum, Bifidobacterium breve; Bifidobacterium catenulatum, Bifidobacterium dentium; Bifidobacterium longum, Bilophila wadsworthia; Burkholderia cepacia; Butyrivibrio fibrisolvens; Campylobacter concisus; Campylobacter curvus; Campylobacter gracilis; Campylobacter jejuni, Campylobacter rectus; Campylobacter showae; Capnocytophaga ochracea; Cedecea spp; Citrobacter freundii; Citrobacter koseri; Clostridium spp.; Desulfomonas pigra; Dysgonomonas spp.; Eikenella corrodens; Enterobacter aerogenes; Enterobacter cloacae; Enterobacter gergoviae; Enterobacter sakazakii; Enterobacter taylorae; Enterococcus spp.; Escherichia coli; Escherichia fergusonii; Escherichia hermannii; Escherichia vulneris; Eubacterium spp.; Finegoldia magnus; Fusobacterium gonidiaformans; Fusobacterium mortiferum; Fusobacterium naviforme; Fusobacterium necrophorum; Fusobacterium nucleatum, Fusobacterium russii; Fusobacterium varium; Gardnerella vaginalis; Gemella morbillorum; Globicatella spp.; Hafnia alvei; Helicobacter spp.; Klebsiella spp.; Lactobacillus acidophilus; Lactobacillus fermentum, Lactobacillus reuteri; Lactobacillus salivarius; Leclercia adecarboxylata; Leminorella spp.; Megasphaera elsdenii; Mitsuokella multiacidus; Mobiluncus curisii; Mobiluncus mulieris; Moellerella wisconsensis; Morganella morganii; Pantoea agglomerans; Pediococcus spp.; Peptoniphilus asaccharolyticus; Peptostreptococcus anaerobus; Peptostreptococcus productus; Porphyromonas asaccharolytica; Proteus mirabilis; Proteus penneri; Proteus vulgaris; Providencia rettgeri; Providencia stuartii; Pseudomonas aeruginosa; Retortamonas intestinalis; Ruminococcus productus; Serratia liquefaciens; Serratia marcescens; Serratia odorifera; Streptococcus agalactiae; Streptococcus anginosus; Streptococcus bovis; Streptococcus constellatus; Streptococcus intermedius; Group C+G Streptococci; Succinivibrio dextrinosolvens; Sutterella spp.; Tissierella praeacuta; Veillonella spp.; Aerobacter spp.; Bacillus anthracis; Bacillus cereus; other Bacillus spp.; Borrelia recurrentis; Brucella spp.; Campylobacter coli; Campylobacter fetus; Campylobacter jejuni, Campylobacter sputorum; Clostridium bifermentans; Clostridium botulinum; Clostridium difficile; Clostridium indolis; Clostridium mangenolii; Clostridium perfringens; Clostridium sordellii; Clostridium sporogenes; Clostridium subterminale; Edwarsiella tarda; Francisella tularensis; Listeria monocytogenes; Mycobacterium bovis; Mycobacterium tuberculosis; Pediococcus spp.; Plesiomonas shigelloides; Rickettsia rickettsiae; Salmonella spp.; Shigella boydii; Shigella dysenteriae; Shigella flexneri; Shigella sonnei; other Spirillum spp.; Streptococcus zooepidemicus; Tropheryma whipplei; Vibrio cholerae; Vibrio fluvialis; Vibrio furnissii; Vibrio hollisae; Vibrio parahaemolyticus; Yersinia enterocolitica; Yersinia pseudotuberculosis; Herpes Simplex virus (1 and 2); Cytomegalovirus; Adenovirus; Orthoreoviruses; Rotaviruses; Alphaviruses; Coronaviruses; Toroviruses; Human metapneumovirus; Vesicular stomatitis virus; Machupo virus; Junin virus; Poliovirus; Coxsackieviruses; Echoviruses; Hepatitis A virus; Noroviruses and other Caliciviruses; Astroviruses; Picobirnaviruses; or Hepatitis E virus.

In alternative aspects, the invention may involve the treatment of an immune dysregulation with formulations of an artificial repertoire of mammalian PRR agonists that recapitulates a distinct portion of a PRR agonist signature of a microbial mammalian pathogen that is a common small and larger bowel pathogens, for example: Escherichia coli, Clostridium difficile, Bacteroides fragilis, Bacteroides vulgatus, Bacteroides thetaiotaomicron, Clostridium perfringens, Salmonella enteriditis, Yersinia enterocolitica, Shigella flexneri; adenoviruses, astroviruses, caliciviruses, noroviruses, rotaviruses, and cytomegalovirus.

In selected embodiments, the invention involves diagnostic steps to assess a patient's previous exposure to an organism. For example, the diagnostic steps may include taking a medical history of exposure to selected pathogens, and/or evaluating a patient's immune response to a selected pathogen. For example, a serology test may be conducted to detect antibodies to selected pathogens in a patient's sera. In connection with this aspect of the invention, antigenic determinants of a selected pathogen may be chosen for use in an immunogenic composition on a selected patient based on a diagnostic indication that the patient has had one or more prior exposure(s) to the pathogen, for example by virtue of the presence of antibodies to antigenic determinants of that pathogen in the patient's sera.

In further selected embodiments, the invention involves diagnostic steps to assess a patient's immunological response to treatment with a selected immunogenic composition. For example, the diagnostic steps may include evaluating a patient's immune response to the immunological determinants of that immunogenic composition, for example using a serological test to detect antibodies to those immunogenic determinants. In connection with this aspect of the invention, a treatment with a selected immunogenic composition may be continued if the evaluation indicates that there is an active immunological response to the immunogenic determinants of that composition, and the treatment may be discontinued, and an alternative treatment with a different immunogenic composition may be initiated, if the evaluation indicates that there is not a sufficiently active immunological response to the immunogenic determinants of the immunogenic composition.

In some embodiments, pre-exposure of an organism to a microbial pathogen may be used to potentiate subsequent SSI efficacy. For example, pre-exposure to K. pneumoniae may, in some embodiments, induce tissue-specific immunologic memory, for example an innate immunological memory, that facilitates tumour cytolysis, particularly in combination with a cytotoxic adoptive immune cell therapy.

SSI and adoptive cell treatments may for example be combined with additional components that potentiate a cancer antigen response. A cancer antigen may for example be admixed with an SSI. The adoptive immune cell therapy may in turn be targeted to the antigen admixed with the SSI.

Microbial components may be formulated as SSTs, containing PRR ligands derived from microbial fractions such as: bacterial outer membrane (for example from Gram negative spp.); bacterial inner membrane; the pellet of a gradient centrifugation (for example from a sucrose gradient); chromosomal DNA; a capsular glycoprotein fraction; or, a peptidoglycan fraction, such as peptidoglycan ghosts. In alternative embodiments, engineered or recombinant organisms may be used in SSTs, in which genes involved in pathways relevant to particular cellular fractions have been modified, in particular genes involved in determining the composition of the foregoing fractions.

For cell fraction preparations, bacteria may for example be grown and heat-inactivated. Cell fractions may for example be resuspended in sterile saline+0.4% phenol. Inner membranes may for example be collected using the 2-step sucrose density gradient, as for example described in Methods in Enzymology, Vol 125:309-328, 1986. The bacterial pellet obtained after cultivation of 250 mls of cells may be resuspended in 20% sucrose, 10 mM Tris-HCl pH 8.0 and 50 ug/ml DNase 1. Cells may be incubated at 23° C. for 10 min. Cells may then be placed on ice and lysed two times through a French pressure cell at 15,000 psi; unbroken cells may be removed by centrifugation at 5,000×g for 10 min at 4° C. Supernatants may be layered onto a 2-step sucrose gradient (60% and 70%) and centrifuged in a SW28 swinging bucket rotor at 23,000 rpm for 18 hours at a temperature of 4° C. The inner membranes may be collected at the junction between the 20% and 60% sucrose. Sucrose may be diluted to below 20% with sterile distilled water and the membranes may be pelleted in an ultracentrifuge at 41,000 rpm at 4° C. for 1 hour. The inner membranes may be washed once with sterile water, and then resuspended in sterile saline+0.4% phenol. Crude outer membrane preparations may also be collected from the junction between the 60% and 70% sucrose gradient steps.

Chromosomal DNA, for example for Klebsiella pneumoniae, may be prepared using a Qiagen Blood and Tissue midi kit. Cells from 15 or 40 mls of broth culture from each strain may be harvested. The manufacture's protocol for purification of total DNA may then be followed.

An SSI may be co-formulated with or co-administered with additional therapeutic components. One class of additional therapeutic components comprises molecules or compositions for activating or recruiting innate immune cells, and these include:
GMCSF, for example in an amount that synergistically recruits and promotes the production of neutrophils and potentiates the SSI-induced innate immune response.
Vitamin D, for example in an amount that is effective to differentiate and activate monocytes and play a role in regulating innate immune function. In alternative embodiments, the vitamin D used in conjunction with SSTs may for example be one or more of vitamin D₃, D₂ or calcitriol (1,25-dihydroxycholecalciferol). In some embodiments, vitamin D₃ and/or D₂ may for example be given locally at a dosage that is effective to provide a locally effective amount of calcitriol at the site of SSI and vitamin D administration. For example, vitamin D precursors (D₃ and/or D₂) may be administered in an amount that is locally effective once it is converted into the calcitriol active form by local monocytes and/or macrophages (expressing CYP27B1) at the site of administration. In alternative embodiments, calcitriol may be administered in dose that is locally effective at the site of SSI administration, and this may for example be dose that is less than the dose required for other systemic effects.

An additional class of therapeutic components for co-formulation or co-administration comprise molecules or compositions that relieve immunosuppression:
NOHA (N(omega)-hydroxy-nor-L-arginine), an Arginase inhibitor—Arginase degrades arginine needed for immune activation. NOHA may for example be used in an amount effective to relieve immune suppression by making available free arginine.
Alpha1 antitrypsin—for example in an amount effective to relieve immune suppression mediated by neutrophils secreting proteases.

An additional class of therapeutic components for co-formulation or co-administration comprise molecules or compositions that prevent oxidative damage and improve immune function under stress:
Glutathione and other antioxidants.

An additional class of therapeutic components for co-formulation or co-administration comprise co-stimulatory molecules for innate cytotoxic lymphocytes (for example for anticancer treatments):
Phospho-antigens (isoprenoid molecules, such as isopentenyl pyrophosphate)—recognized by human peripheral blood Vγ9Vð2 T cells which play a central role in anticancer responses, for example in amounts effective for activating and differentiating monocytes working in concert with NK cells to target both solid and liquid cancers. In exemplary embodiments, it has been found that SSTs in co-formulation or co-administration with zoledronate increase markers of activation, for example CD25 and CD69, on human peripheral blood Vγ9Vð2 T cells.
Glycolipid molecules recognized by Type I NKT cells (such as synthetic a-galactosylceramide).

SSTs may for example be administered for systemic distribution. A KPN SSI administered subcutaneously in a murine model, using cyanine dye (Cy5.5) labeled whole killed KPN cells and optical in-vivo dorsal and ventral whole-body imaging, revealed systemic distribution with highest concentrations seen at the new sites of injection and, surprisingly, at previous sites of injection. This provides an illustration of preferential SSI delivery/retention at sites of inflammation following systemic dispersal of locally administered formulations. The distribution of SSI in organs after 24 hours showed a preferential accumulation of KPN SSI in the lungs, compared to the heart and the spleen.

In select embodiments, SSTs can be administered directly to cancerous tissues, for example at the site of surgical resection of a cancer. For example, an SSI may be applied topically to a melanoma in the skin or to the site of a surgical excision of a skin melanoma.

Clinical data has shown the efficacy of SSTs acting to down-regulate PD1 and PDL1 expression in neoplastic disease. Accordingly, PD1 and PDL1 may be sued as markers of SSI efficacy. In addition, SSTs may be formulated and administered in a dosage regime that is effective in a target organ or tissue to mediate increased expression of one or more granzyme or perforin, such as of granzyme A, granzyme B, and perforin.

A variety of PRR receptors may be used as the targets for alternative SSTs.

TABLE 8
List of PRRs stimulated by select SSIs, including QBKPN, QBECO and QBSAU
(Staphylococus aureus SSI). Where a PRR is “Optional”, this indicates
that some embodiments may be designed to include agonists for the specified PRR.

| | | | | |
|---|---|---|---|---|
| Pattern | | | | |
| Recognition | | | | |
| Receptor | Major Agonists | QBECO | QBKPN | QBSAU |

TLRs (Toll-Like Receptors)

| | | | | |
|---|---|---|---|---|
| TLR1 | Triacyl lipoprotein/peptidoglycan | Yes | Yes | Yes |
| TLR2 | Glycolipds, Lipoprotein, | Yes | Yes | Yes |
| | lipopeptides, lipoteichoic acid, | | | |
| | others | | | |
| TLR3 | dsRNA (viral) | No | No | No |
| TLR4 | Lipopolysaccharide (LPS), heat | Yes | Yes | No |
| | shock proteins, others | | | |
| TLR5 | Flagellin, Profilin | Yes | No | No |
| TLR6 | Diacyl lipoprotein | Yes | Yes | Yes |
| TLR7 | ssRNA | No | No | No |
| TLR9 | CpG-DNA | Yes | Yes | Yes |
| TLR10 | Unclear | Optional | Optional | Optional |

CLR (C-Type Lectin Receptors) (PMID 21616435)

| | | | | |
|---|---|---|---|---|
| Mannose | Mannose, N-acetylglucosamine | Optional | Optional | Optional |
| Receptor (MR) | and fucose on glycans | | | |
| DEC-205 | Promiscuous antigen receptor- | Optional | Optional | Optional |
| | Class B CpG-DNA (Lahoud et | | | |
| | al. 2012. PNAS) | | | |
| Macrophage | α- or β-N-acetylgalactosamine | Optional | Optional | Optional |
| galactose-type | (GalNAc, Tn) residues of N- | | | |
| lectin (MGL) | and O-glycans carried by | | | |
| | glycoproteins and/or | | | |
| | glycosphingolipids (PMID | | | |
| | 15802303) | | | |
| DC-SIGN (CD- | High-mannose-containing | Optional | Optional | Optional |
| 209) | glycoproteins | | | |
| Langerin (CD207) | Similar to CD-209 | Optional | Optional | Optional |
| Mannose Binding | Mannose and N- | Optional | Optional | Optional |
| Lectin (MBL) | acetylucosamine | | | |
| Myeloid DAP12- | Unclear, dengue viral particles | Optional | Optional | Optional |
| associating lectin | (PMC3204838) | | | |
| (MDL-1/CLEC5A) | | | | |
| Dectin1/CLEC7A | B glucans on fungi, | Optional | Optional | Optional |
| | mycobacteria | | | |
| DNGR1/CLEC9A | Actin filaments (no microbial | Optional | Optional | Optional |
| | ligands identified) | | | |
| SIGNR3 | Mycobacterium tuberculosis | Optional | Optional | Optional |
| CLEC4B1 | Not Determined | Optional | Optional | Optional |
| CLEC4B2 | Not Determined | Optional | Optional | Optional |
| CLEC2 | Endogenous (prodoplanin), | Optional | Optional | Optional |
| | snake venom, HIV | | | |
| CLEC12B | Not Determined | Optional | Optional | Optional |
| CLEC12A | Not Determined | Optional | Optional | Optional |
| DCIR/CLEC4A | HIV-1 | Optional | Optional | Optional |
| Dectin 2/CLEC6A | Mannose-type carbohydrates | Optional | Optional | Optional |
| CLEC4C | Unclear | Optional | Optional | Optional |
| CLEC4E (Mincle) | Fungal a-mannose and others | Optional | Optional | Optional |

NLR (Nod-Like Receptors)

| | | | | |
|---|---|---|---|---|
| NOD1 | diaminopimelatic acid (DAP)- | Optional | Optional | Optional |
| | containing muropeptide | | | |
| NOD2 | muramyl dipeptide (MDP) | Yes | Yes | Optional |
| | moieties universal to all bacterial | | | |
| | peptidoglycan | | | |
| NLRC3 (NOD3) | Cytosolic DNA, cyclic di-GMP, | Optional | Optional | Optional |
| | DNA viruses (PMID 24560620) | | | |
| | This is an inhibitory PRR. | | | |
| NLRC4 (NOD4) | Flagellin, components of the | Optional | Optional | Optional |
| | type three secretion system, | | | |
| | others | | | |
| NLRC6 | Unclear | Optional | Optional | Optional |
| NLRX1 (NOD5) | Unclear | Optional | Optional | Optional |
| NALP1-14 | Pathway unclear (Anthrax and | Optional | Optional | Optional |
| | muramyldipeptide for NALP1) | | | |
| NAIP | Unclear | Optional | Optional | Optional |
| CIITA (NLRA) | Unclear (does not directly bind | Optional | Optional | Optional |
| | DNA) | | | |

RLR (Rig-1 Like Receptors)

| | | | | |
|---|---|---|---|---|
| RIG-1 | dsRNA (viral), maybe bacterial | Optional | Optional | Optional |
| MDA5 | dsRNA (viral) | Optional | Optional | Optional |
| LGP2 | dsRNA (viral) | Optional | Optional | Optional |
| Others | | | | |
| DAI (DNA- | DNA | Optional | Optional | Optional |
| dependent | | | | |
| activator of IRFs) | | | | |
| (PMID 20098460) | | | | |
| AIM2 (PMID | dsDNA | Optional | Optional | Optional |
| 20098460) | | | | |
| Caspase 11 | LPS | Optional | Optional | Optional |
| (PMID 25145754) | | | | |
| LBP | LPS | Optional | Optional | Optional |
| (Lipopolysaccharide | | | | |
| Binding Protein | | | | |
| CD14 | LPS | Optional | Optional | Optional |
| Scavenger | LPS | Optional | Optional | Optional |
| Receptors | | | | |
| Beta2 Integrins | LPS | Optional | Optional | Optional |
| Peptidoglycan | Peptidoglycan | Minor | Minor | Major |
| receptor proteins | | | | |
| (4 different | | | | |
| receptors) | | | | |

TABLE 9
PRR agonists in select fractionated SSIs, particularly in the DNA fractions.
DNA Fractions

| Component | QBECO | QBKPN |
|---|---|---|
| DNA | TLR9 | TLR9 |
| | AIM2 | AIM2 |
| | DAI | DAI |
| | RIG-1 | RIG-1 |
| | DEC205 | DEC205 |
| | NLRC3 | NLRC3 |

TABLE 10
PRR agonists in select fractionated SSIs, particularly
in the outer membrane fractions.

| Component | QBECO | QBKPN |
|---|---|---|
| LPS | TLR4 | TLR4 |
| | LBP | LBP |
| | CD14 | CD14 |
| | Caspase 11 | Caspase 11 |
| | Other | Other |
| | Scavenger | Scavenger |
| | Receptors | Receptors |
| Lipoprotein | TLR1 | TLR1 |
| | TLR2 | TLR2 |
| | TLR6 | TLR6 |
| Flagellin | TLR5 | N/A |
| | NOD4 | NOD4 |
| Peptidoglycan | NOD2 | NOD2 |
| Capsule | N/A | TLRs and CLRs |
| Other | Collection of | |
| | CLRs | |

Accordingly, in select embodiments, SSI therapies are provided that target a select subset of PRRs, using microbial PRR agonists derived from microbial pathogens of a target tissue. For example, an immunogenic composition is provided that comprises microbial agonists for at least a select number of distinct PRRs, for use so as to illicit an innate response in a target tissue, wherein the PRR agonists are microbial components from a single species of microbe that is selectively pathogenic in the target tissue. The number of distinct PRRs targeted by the agonists may for example be a number from 5 to 25, or at least a number within that range of integers, for example at least 5, 6, 7, 8, 9, 10, 11, 12, 13, 14, 15, 16, 17, 18, 19, 20, 21, 22, 23, 24, 25 etc. The distinct PRRs may for example be selected from the PRRs set out in Tables 8, 9 and/or 10.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification, and all documents cited in such documents and publications, are hereby incorporated herein by reference as if each individual publication were specifically and individually indicated to be incorporated by reference herein and as though fully set forth herein. The invention includes all embodiments and variations substantially as described herein and with reference to the examples and drawings. In some embodiments, the invention excludes steps that involve medical or surgical treatment.

### General Codes and Abbreviations

| | |
|---|---|
| Mean; MEAN | Average |
| SD; S.D.; STAND DEV; | Standard deviation |
| STANDARD DEV; sd; | |
| STD.DEV | |
| SE; S.E. | Standard error |
| —; NA | No value; not applicable; |
| | not present |
| #; N; No. | Number |
| Obs. | Observations |
| IPD | Immediate postdose |
| PD | Postdose |
| a.m. | Ante meridian |
| p.m. | Post meridian |
| M | Male |
| F | Female |
| ID | Identification |
| S.C. | Subcutaneous |
| I.V. or IV or i.v. or iv | Intra-venous |
| I.P. or IP or i.p. or ip | Intraperitoneal |
| K. pneumoniae, KPN | Klebsiella pneumoniae |
| QBKPN, KP SSI | Klebsiella spp. SSI, a |
| | formulation comprising |
| | PRR ligands of whole |
| | killed Klebsiella spp cells |
| | in aqueous suspension of |
| | a pathogenic Klebsiella |
| | clinical isolate variously |
| | identified as |
| | K. pneumonia or K. variicola |
| | (Klebsiella phylogroup III) |
| QBECO, EC SSI | E. coli SSI, a formulation |
| | comprising PRR ligands |
| | of whole killed E. coli cells |
| | in aqueous suspension of |
| | an enteropathogenic |
| | E. coli clinical isolate. |
| QBSAU, SA SSI | Staphylococcus aureus |
| | SSI, a formulation |
| | comprising PRR ligands |
| | of whole killed S. aureus |
| | cells in aqueous |
| | suspension of a |
| | pathogenic S. aureus |
| | clinical isolate. |
| SSI | Site specific |
| | Immunomodulator, a PRR |
| | ligand formulation |
| UT | Un-treated |
| Ns | Not significant |
| B6 | C57BL/6 mice |
| G, g | Gram |
| KG, kg | Kilogram |
| MG, mg | Milligram |
| ML, mL | Milliliter |
| UL, μL | Microliter |
| HR, hr | Hours |
| min | Minute |

### Examples

### SSIs Ameliorate Post-Surgical Immune Suppression/Metastasis

As illustrated in the following Examples, in a murine lung and colon/liver cancer metastasis models, perioperative SSI treatments markedly reduced metastasis and relieved post-surgical immune suppression. Exemplary data illustrate the therapeutic modulation of multiple immune pathways (MHCII, CD96, CD69, IFNγ) and the recruitment of multiple important innate immune cells (NK cells, monocytes and neutrophils).

### SSI Treatment Reduces Post-Surgical Lung Metastases

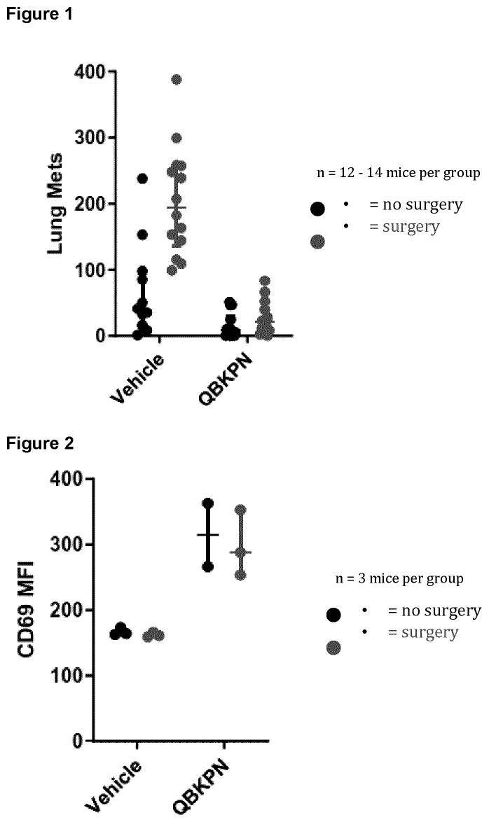

Mice were pre-treated with QBKPN or vehicle (placebo) every second day for 8 days prior to i.v. tumor injection+/−surgery (laparotomy and nephrectomy) and 1 day post-i.v tumor injection. The total number of lung metastases were quantified 3 days post-surgery. As illustrated in FIG. 1, QBKPN SSI treatment substantially reduced lung metastases with and without surgery.

### QBKPN Increases NK Cell Activation

Mice were pre-treated with QBKPN or vehicle (placebo) every second day for 8 days prior to i.v. tumor injection+/−surgery (laparotomy and nephrectomy) and 1 post-i.v tumor injection. NK cell activation in the spleen was quantified by cells expressing CD69, 3 days post surgery. As illustrated in FIG. 2, QBKPN treatment increases NK cell activation, as measured by CD69 expression.

### QBKPN Decreases NK Cell Inhibition

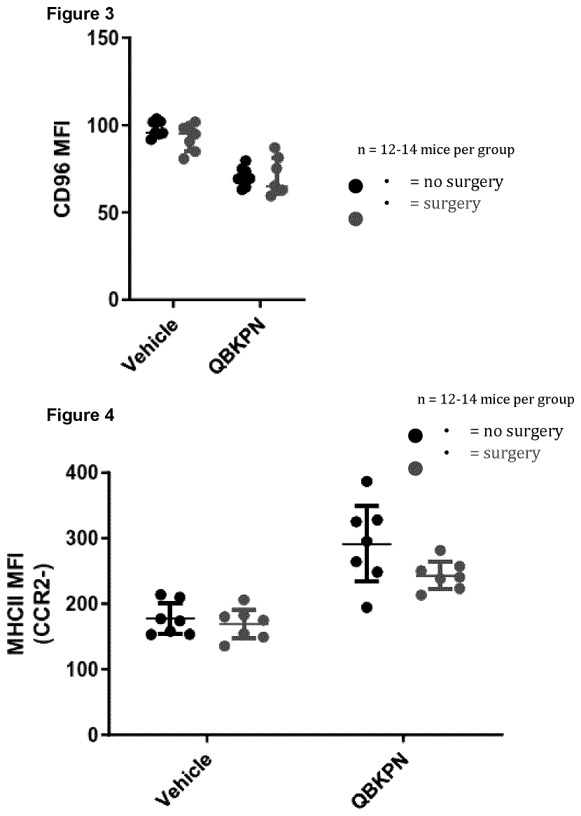

Mice were pre-treated with QBKPN or vehicle (placebo) every second day for 8 days prior to i.v. tumor injection+/−surgery (laparotomy and nephrectomy) and 1 post-i.v tumor injection. NK cell activation in the spleen was quantified by cells expressing CD96, 3 days post surgery. As illustrated in FIG. 3, QBKPN treatment reduces expression of the NK cell checkpoint inhibitor, CD96, resulting in decreased NK cell inhibition i.e., NK cell activation.

### QBKPN Increases Monocyte/Macrophage Activation

Mice were pre-treated with QBKPN or vehicle (placebo) every second day for 8 days prior to i.v. tumor injection+/−surgery (laparotomy and nephrectomy) and 1 post-i.v tumor injection. Monocyte/macrophage activation in the spleen was quantified by cells expressing MHCII, 3 days post surgery. As illustrated in FIG. 4, QBKPN treatment enhances immune surveillance and M1 monocyte/macrophage polarization (activation) as measured by increase in MHCII expression.

### QBKPN Increases Neutrophils

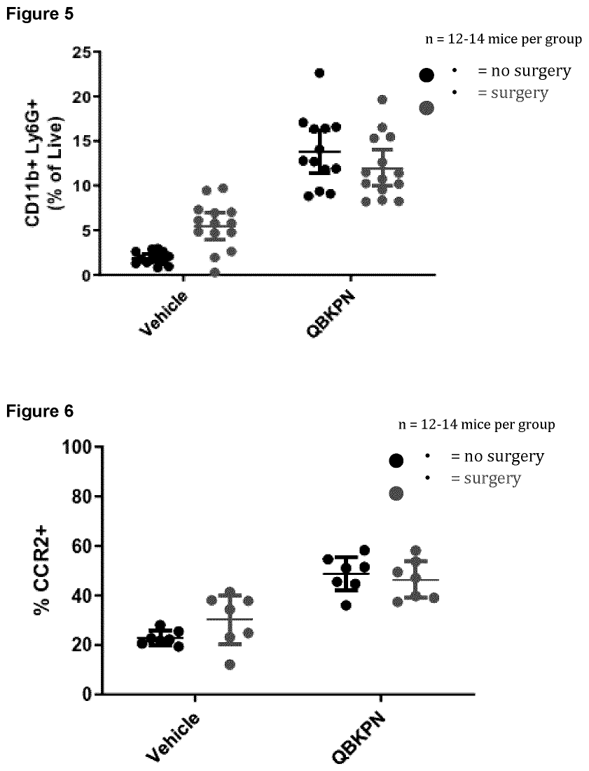

Mice were pre-treated with QBKPN or vehicle (placebo) every second day for 8 days prior to i.v. tumor injection+/−surgery (laparotomy and nephrectomy) and 1 day post-i.v tumor injection. Neutrophil cell counts (CD11b+Ly6G+) from the spleen were quantified as % of live cells, 3 days pos-surgery. As illustrated in FIG. 5, QBKPN treatment increases myelopoiesis (neutrophil counts, as indicative of myelopoiesis which is also associated with increased monocyte counts in SSI therapies).

### QBKPN Increases Immune Cell Recruitment

Mice were pre-treated with QBKPN or vehicle (placebo) every second day for 8 days prior to i.v. tumor injection+/−surgery (laparotomy and nephrectomy) and 1 day post-i.v tumor inj. Splenic monocyte/macrophage CCR2 receptor expression was measured 3 days post-surgery. As illustrated in FIG. 6, QBKPN treatment upregulates CCR2 chemokine receptor expression on immune cells. Previous data demonstrates QBKPN increases lung-specific release of chemokines, resulting in recruitment of activated immune cells expressing CCR2 to the lungs.

### QBKPN Increases Immune Activation

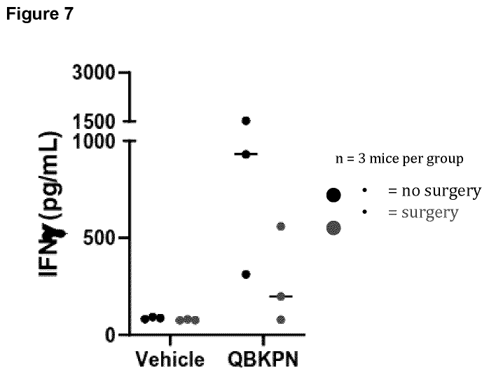

Mice were pre-treated with QBKPN or vehicle (placebo) every second day for 8 days prior to i.v. tumor injection+/−surgery (laparotomy and nephrectomy) and 1 day post-i.v tumor injection. Immune activation was quantified on Day 1 post-surgery by stimulating whole blood with a cytokine cocktail for 12 hours and measuring IFNγ levels. As illustrated in FIG. 7, QBKPN treatment increases immune activation, as measured by IFNγ release, relieving immune suppression post-surgery.

### QBECO Inhibits Liver Perioperative Metastasis in Colon Cancer

In this murine model, all animals receive surgery as an act of tumor seeding. The act of injecting tumor cells via the portal vein involves a laparotomy with significant anatomical manipulation and operative time, with attendant significant surgical stress. Female C57Bl/6 mice were injected with either vehicle (PBS) or SSI QBECO subcutaneously for 4 doses, every second day, up to and excluding the day of MC38-GFP tumor cell implantation via the portal vein at a concentration of 10\hat{ }6 cells/20 uL. One day post tumor cell implantation, the mice continued to receive either vehicle or SSI for 7 doses, every second day. Mice were sacrificed two days after the last treatment injection.

The animals were perfused via the portal vein, and the livers were then excised and fixed in 10% formalin for 48 hours prior to paraffin-embedding. 10 sections per liver were acquired, at 100 um apart. Each section is cut at 4 um thick and hematoxylin and eosin (H&E) stain was performed.

Liver images were scanned with Zeiss AxioScan and processed using FIJI ImageJ software. Tumor burden was calculated as sum of tumor areas (um\hat{ }2) from all sections from each liver, divided by sum of total liver areas (um\hat{ }2) from all sections, expressed as a percentage.

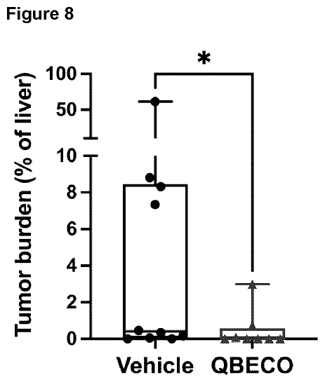

Statistical significance was determined using the Mann-Whitney test, where an asterisk * denotes p<0.05. Individual data points were plotted on a box & whisker graph, with the whiskers showing min to max points. FIG. 8 illustrates the results, showing the striking efficacy of QBECO in reducing tumor burden (% of liver) in this model of perioperative liver metastasis.

### SSIs Modulate Perioperative NK Cell Function

Female C57Bl/6 mice were injected with either vehicle (PBS), or SSI subcutaneously for 6 doses, every second day, up to and including the day of surgery (a laparotomy and nephrectomy). Mice were sacrificed one day post surgery.

Immunomodulatory effects were assayed by NKVue (IFNg production by NK cells), as follows. A cardiac puncture was performed to retrieve whole blood into a heparin-coated tube. Immediately, blood was incubated with Promoca (a proprietary NK stimulation mix used with the NKVue assay) for 12 hours at 37 C. At the end of incubation, supernatant (plasma) was collected and frozen at −80 C until running the ELISA (stimulated series). An unstimulated series of plasma was also obtained by spinning out the blood immediately after removal from the mice. IFNg production by NK cells from both series were measured using the NKVue ELISA kit.

Immunomodulatory effects were also assayed in a NK cytotoxicity assay, as follows. NK cells were isolated using the EasySep Mouse NK Cell Isolation Kit (StemCell) from spleens. Target cells (Yac-1) were labelled with cell trace violet (CTV) prior to incubation with NK cells at ratios of 27:1 (NK cell to Yac-1), 9:1, 3:1, and 1:1 to examine the NK cell cytotoxic capability. The cells were cultured for 4 h at 37 C. Thereafter, cells were stained with viability dye (Zombie NIR from BioLegend) and fixed with 1% PFA for analysis on the BD Fortessa the next day.

NK Vue assay results in the context of QBSAU treatment in the peri-operative period demonstrated that QBSAU treatment restores NK cell function (as measured by IFN-gamma production) in the post-operative period as compared to NK cell paralysis (minimal IFN-gamma production) in a control group of mice treated with vehicle. This data reflects the efficacy of a QBSAU SSI treatment in eliciting therapeutic immunomodulating effects in common with the therapeutic mechanism of action of other SSTs. Immunomodulatory effects evidenced in the NK cytotoxicity assay were also similar using a range of SSTs, QBECO/QBSAU/QBKPN.

This data accordingly Illustrates the ability of a range of SSTs, in addition to QBECO and QBKPN, to have a similar perioperative therapeutic effect in modulating an immune response in a target tissue in a way that is effective to treat residual disease and thereby treat cancer, for example by reducing peri-operative metastases, as demonstrated with QBKPN and QBECO.

Previous preclinical studies have demonstrated that NK cells play an important role in SSI efficacy and that SSI's ‘train’ NK cells to improve NK cell functionality, including increasing NK cell IFN-gamma response to infectious and non-infectious threats (such as cancer). This SSI induced ‘training’ of NK cell function, as measured by IFN-gamma production upon NK cell stimulation (e.g. in an NK Vue assay), is thought to play a primary role in SSI induced reduction of pen-operative metastases, since, without SSI treatment, NK cell function is ‘paralyzed’ in the post-operative period (with minimal IFN-gamma production in the NK Vue assay) but with SSI treatment, NK cell function is therapeutically modulated (as measured by restoration of IFN-gamma production), enabling NK cell clearance/amelioration of metastatic lesions.

### QBECO for Treating Liver Metastasis in Colon Cancer

This is an exemplary protocol for treating liver metastasis following primary colon cancer surgery, involving the use of circulating tumor DNA (ctDNA) as a diagnostic. Surgery to remove primary colon cancer tumors is often curative. However, in some patients, residual disease progresses in the form of metastasis to the liver. In some of these cases, surgery is an option for resection of metastatic colon cancer tumors situated in the liver. Due to post-operative immune suppression, such patients may be at risk for additional metastasis or growth of any residual cancer cells during or post-surgery. An E. coli based SSI, QBECO, may be used in the perioperative period surrounding surgery to prevent or remove these residual cancer cells or metastases. A useful adjunct to this SSI therapy is monitoring of ctDNA, in particular because the presence of residual disease is indicated if ctDNA is detected in a liquid biopsy, for example being detected as long as 1, 2, 3 weeksor one, 1, 2, 3, 4, 5, 6, 7, 8, 9, 10, 11 or 12 months following the liver surgery (Cescon, D. W., Bratman, S., Chan, S. M. et al. Circulating tumor DNA and liquid biopsy in oncology. Nat Cancer 1, 276-290 (2020) DOI 10.1038/s4318-020-0043-5; Daniel Andersson, Helena Kristiansson, Mikael Kubista & Anders Stahlberg (2021) Ultrasensitive circulating tumor DNA analysis enables precision medicine: experimental workflow considerations, Expert Review of Molecular Diagnostics, DOI: 10.1080/14737159.2021.1889371).

Patients presenting with liver metastasis with or without prior colon cancer surgery may be treated with QBECO prior to a date scheduled for surgery to remove liver tumors. The treatment may for example be every other day for 1, 2, 3 weeks or 1, 2, 3, 4, 5, 6, 7, 8, 9, 10, 11 or 12 months prior to surgery, and/or for 1, 2, 3 weeks or 1, 2, 3, 4, 5, 6, 7, 8, 9, 10, 11 or 12 months following surgery. During this perioperative period, liquid biopsies may be monitored for ctDNA to provide a prognostic indicator of residual disease.

Other assays that may be used in perioperative SSI therapies include immune-assays for trained innate immunity, NK Vue™ assays, and metabolic assays.

### REFERENCES

Lachmann G, von Haefen C, Kurth J, Yuerek F, Spies C. Innate immunity recovers earlier than acquired immunity during severe postoperative immunosuppression. Int J Med Sci 2018; 15(1):1-9. doi:10.7150/ijms.21433).
Bartal I, Melamed R, Greenfeld K, Atzil S, Glasner A, Domankevich V. et al. Immune perturbations in patients along the perioperative period: alterations in cell surface markers and leukocyte subtypes before and after surgery. Brain, behavior, and immunity. 2010; 24:376-86
Goldfarb Y, Sorski L, Benish M, Levi B, Melamed R, Ben-Eliyahu S. Improving postoperative immune status and resistance to cancer metastasis: a combined perioperative approach of immunostimulation and prevention of excessive surgical stress responses. Annals of surgery. 2011; 253:798-810.
Levi B, Metzner P, Goldfarb Y, Sorski L, Shaashua L, Melamed R, Rosenne E, Page G G, Ben-Eliyahu S. Stress impairs the efficacy of immune stimulation by CpG-C: Potential neuroendocrine mediating mechanisms and significance to tumor metastasis and the perioperative period. Brain Behav Immun. 2016 August; 56:209-220. doi: 10.1016/j.bbi.2016.02.025. Epub 2016 Mar. 2.
Benish M, Bartal I, Goldfarb Y, Levi B, Avraham R, Raz A, Ben-Eliyahu S. Perioperative use of beta-blockers and COX-2 inhibitors may improve immune competence and reduce the risk of tumor metastasis. Ann Surg Oncol. 2008 July, 15(7):2042-52. doi: 10.1245/s10434-008-9890-5. Epub 2008 Apr. 9.
Metzner P, Sandbank E, Neeman E, Zmora O, Gottumukkala V, Ben-Eliyahu S. Harnessing cancer immunotherapy during the unexploited immediate perioperative period. Nat Rev Clin Oncol. 2020 May; 17(5):313-326. doi: 10.1038/s41571-019-0319-9. Epub 2020 Feb. 17.
Sundararaj S C, Thomas M V, Dziubla T D, Puleo D A. Bioerodible system for sequential release of multiple drugs. Acta Biomater. 2014, 10(1):115-125.
Han Lu, Peng Ke, Qiu Li-Ying, Li Meng, Ruan Jing-Hua, He Li-Li, Yuan Zhi-Xiang. Hitchhiking on Controlled-Release Drug Delivery Systems: Opportunities and Challenges for Cancer Vaccines. Frontiers in Pharmacology, 2021, vol 12.

1. Use of an effective amount of an immunogenic composition to treat a cancer in a mammalian subject, wherein:
the cancer forms a tumor in a target tissue, and/or the cancer is characterized by a potential to metastasize to the target tissue;
the composition is for use in combination with surgical removal of the tumor on a surgery date;
the composition comprises a repertoire of mammalian pattern recognition receptor (PRR) ligands that recapitulates at least a portion of a PRR agonist signature of a microbial mammalian pathogen that is pathogenic in the target tissue, wherein the repertoire of mammalian PRR ligands are formulated together in a therapeutic vehicle for combined presentation following administration to the mammalian subject, and the composition comprises components of the microbial mammalian pathogen that are ligands for at least 5 distinct mammalian PRRs; and,
the immunogenic composition is for use in a perioperative period that is within one month of the surgery date so as to modulate an immune response in the target tissue that is effective to treat residual disease and thereby treat the cancer.

2. A method for treating a cancer in a mammalian subject, wherein the cancer forms a tumor in a target tissue, and/or the cancer is characterized by a potential to metastasize to the target tissue, wherein the subject undergoes surgical removal of the tumor on a surgery date, the treatment comprising:
administering to the subject an effective amount of an immunogenic composition during a perioperative period that is within one month of the surgery date;
wherein the composition comprises an artificial repertoire of mammalian pattern recognition receptor (PRR) ligands that recapitulates at least a portion of a PRR agonist signature of a microbial mammalian pathogen that is pathogenic in the target tissue, wherein the repertoire of mammalian PRR ligands are formulated together in a therapeutic vehicle for combined presentation following administration to the mammalian subject, and the composition comprises components of the microbial mammalian pathogen that are ligands for at least 5 distinct mammalian PRRs; and,
the immunogenic composition is administered so as to modulate an immune response in the target tissue that is effective to treat residual disease and thereby treat the cancer.

3. The use or method of claim 1 or 2, wherein the PRR ligands are PRR agonists.

4. The use or method of any one of claims 1 to 3, wherein the immunogenic composition modulates an innate immune response in the target tissue.

5. The use or method of any one of claims 1 to 4, wherein the repertoire of mammalian pattern recognition receptors is an artificial repertoire and the portion of the PRR agonist signature is a distinct portion that is different from any native PRR ligand signature of the microbial mammalian pathogen.

6. The use or method of any one of claims 1 to 5, wherein the tumor is removed from the target tissue.

7. The use or method according to any one of claims 1 to 6, wherein the subject is a mouse, cat, dog, horse, rodent or human.

8. The use or method of any one of claims 1 to 7, wherein the therapeutic vehicle comprises a microbial cell, a recombinant microbial cell, a cellular fraction of the recombinant microbial cell, a cellular fraction of the microbial cell, a bacterial outer membrane fraction, a bacterial inner membrane fraction, a pellet from a gradient centrifugation of microbial cell components, microbial chromosomal DNA, a microparticle or a liposome, each comprising components of the microbial mammalian pathogen that provide the PRR agonists that together make up the repertoire of PRR agonists.

9. The use or method of claim 8, wherein the recombinant microbe comprises a recombinant gene encoding a component of at least one of the PRR agonists.

10. The use or method of claim 8 or 9, wherein the therapeutic vehicle comprises a whole killed or attenuated microbial cell or recombinant microbial cell.

11. The use or method according to any one of claims 1 to 10, wherein the PRRs and the corresponding PRR ligands are selected from the group consisting of:

| PRR | PRR Ligand |
|---|---|
| TLR2 | Microbial cell wall |
| | components/preparations, Pam2C- |
| | Aca-Benzyl-Murabutide (Pam2C- |
| | conjugated murabutide) |
| TLR3 | Polyadenylic-polyuridylic acid, |
| | Polyinosine-polycytidylic acid |
| TLR4 | Lipopolysaccharide, Monophosphoryl |
| | Lipid A |
| TLR5 | Flagellin |
| TLR7/8 | Single-stranded RNAs, Nucleoside |
| | analogs, |
| | Imidazoquinolines/Thiazoquinolines |
| TLR9 | unmethylated CpG DNA motifs |
| NOD1 | iE-DAP, Acylated iE-DAP, D-gamma- |
| | Glu-mDAP, L-Ala-gamma-D-Glu-mDAP |
| NOD2 | MDP (MurNAc-L-Ala-D-isoGln, |
| | muramyl dipeptide), N-glycolylated |
| | muramyldipeptide, N-Acetyl-muramyl- |
| | L-Alanyl-D-Glutamin-n-butyl-ester, |
| | MurNAc-Ala-D-isoGln-Lys, N- |
| | Acetylmuramyl-L-Alanyl-D- |
| | Isoglutamine (L-D isoform), 6-O- |
| | stearoyl-N-Acetyl-muramyl-L-alanyl-D- |
| | isoglutamine, Pam2C-Aca-Benzyl- |
| | Murabutide, |
| TLR2/NOD2 | Pam2C-conjugated murabutide |
| NOD1/NOD2 | PGN, Pam2C-conjugated murabutide |
| RIG1/MDA5 | 5′ triphosphate double stranded RNA |
| | (18-20mer), |
| | polyriboinosinic: polyribocytidylic acid |
| DAI, LRRFIP1, AIM2, | dsDNA, poly(dA-dT)•poly(dT-dA) |
| RIG1 | |
| Dectin-1 | Beta-glucan peptide, fungal cell wall |
| | preparations |
| Mincle | damaged microbial cells, fungus, yeast |
| | and mycobacteria, Trehalose-6,6- |
| | dibehenate, trehalose-6,6-dimycolate |
| STING | Cyclic dinucleotides (c-di-nucleotides), |
| | xanthenone derivatives, 3′3′-cGAMP, |
| | 2′3′-cGAMP, 2′2′-cGAMP, 2′2′-cGAMP. |
| | c-di-AMP (cyclic di-adenylate |
| | monophosphate), c-di-GMP, c-di-IMP, |
| | c-di-UMP, c-di-AMP |
| RIG-I | PPP-ssRNA (PPP-ssRNA, ssRNA with |
| | a 5′-triphosphate group), RNA with |
| | base pairing and polyl:C |
| MDA5 | Long dsRNA |
| LGP2 | dsRNA |
| DDX41 | B-form DNA and CDNs (cyclic |
| | dinucleotides) |
| DHX9 | DNA, RNA, CpG-A |
| | oligodeoxynucleotids and CpG-B |
| | ODNs |
| DDX3 | Viral RNA |
| DHX36 | DNA, RNA, CpG-A |
| | oligodeoxynucleotids and CpG-B |
| | oligodeoxynucleotids |
| DDX1-DDX21-DDX36 | RNA and polyl:C |
| DDX60 | ssRNA, dsRNA and dsDNA |
| KU70 | DNA |
| cGAS | DNA |
| STING | CDNs (c-di-GMP and c-di-AMP) |
| NOD2 | SSRNA |
| NLRP3 | ssRNA, dsRNA, bacterial mRNA and |
| | oxidized mitochondrial DNA |
| AIM2 | DNA |
| IFI16 | dsDNA |
| LRRFIP1 | B-form DNA, Z-form DNA and dsRNA |
| DAI | DNA |
| IFIT1,2,3 and 5 | PPP-ssRNA |

12. The use or method according to any one of claims 1 to 11, wherein the target tissue and the corresponding microbial mammalian pathogen are selected from the group consisting of:

| Target Tissue | Microbial Mammalian Pathogen |
|---|---|
| Skin | Staphylococcus aureus, Beta hemolytic |
| | streptococci group A, B, C and G, |
| | Corynebacterium diptheriae, Corynebacterium |
| | ulcerans, Pseudomonas aeruginosa |
| | rubeola, rubella, varicella-zoster, echoviruses, |
| | coxsackieviruses, adenovirus, vaccinia, herpes |
| | simplex, parvo B19 |
| Soft tissue (i.e. | Streptococcus pyogenes, Staphylococcus |
| fat and | aureus, Clostridium perfringens, other |
| muscle) | Clostridium spp. |
| (e.g., sarcoma) | influenza, coxsackieviruses |
| Breast | Staphylococcus aureus, Streptococcus pyogenes |
| Lymph nodes: | Staphylococcus aureus, Streptococcus pyogenes |
| head and neck | Epstein-Barr, cytomegalovirus, adenovirus, |
| | measles, rubella, herpes simplex, |
| | coxsackieviruses, varicella-zoster |
| Lymph nodes: | Staphylococcus aureus, Streptococcus pyogenes |
| axillae/arm | measles, rubella, Epstein-Barr, cytomegalovirus, |
| | adenovirus, varicella-zoster |
| Lymph nodes: | viridans streptococci, Peptococcus spp., |
| mediastinal | Peptostreptococcus spp., Bacteroides spp., |
| | Fusobacterium spp., Mycobacterium tuberculosis |
| | measles, rubella, Epstein-Barr, cytomegalovirus, |
| | varicella-zoster, adenovirus |
| Lymph nodes: | Streptococcus pneumoniae, Moraxella |
| pulmonary | catarrhalis, Mycoplasma pneumoniae, Klebsiella |
| hilum | pneumoniae, Haemophilus influenza, |
| | Chlamydophila pneumoniae, Bordetella |
| | pertussis, Mycobacterium tuberculosis |
| | influenza, adenovirus, rhinovirus, coronavirus, |
| | parainfluenza, respiratory syncytial virus, human |
| | metapneumovirus, coxsackievirus |
| Lymph nodes: | Yersinia enterocolitica, Yersinia |
| intra- | pseudotuberculosis, Salmonella spp., |
| abdominal | Streptococcus pyogenes, Escherichia coli, |
| | Staphylococcus aureus, Mycobacterium |
| | tuberculosis |
| | measles, rubella, Epstein-Barr, cytomegalovirus, |
| | varicella-zoster, adenovirus, influenza, |
| | coxsackieviruses |
| Lymph nodes: | Staphylococcus aureus, Streptococcus pyogenes |
| inguinal/leg | measles, rubella, Epstein-Barr, cytomegalovirus, |
| | herpes simplex |
| Hematological | Staphylococcus aureus, Streptococcus |
| (e.g. | pyogenes, coagulase-negative staphylococci, |
| leukemias, | Enterococcus spp., Escherichia coli, Klebsiella |
| multiple | spp., Enterobacter spp., Proteus spp., |
| myeloma) | Pseudomonas aeruginosa, Bacteroides fragilis, |
| | Streptococcus pneumoniae, group B streptococci |
| | rubeola, rubella, varicella-zoster, echoviruses, |
| | coxsackieviruses, adenovirus, Epstein-Barr, |
| | cytomegalovirus, herpes simplex |
| Bone | Staphylococcus aureus, coagulase-negative |
| | staphylococci, Streptococcus pyogenes, |
| | Streptococcus pneumoniae, Streptococcus |
| | agalactiae, other streptococci spp., Escherichia |
| | coli, Pseudomonas spp., Enterobacter spp., |
| | Proteus spp., Serratia spp. |
| | parvovirus B19, rubella, hepatitis B |
| Joint | Staphylococcus aureus, coagulase-negative |
| | staphylococci, Streptococcus pyogenes, |
| | Streptococcus pneumoniae, Streptococcus |
| | agalactiae, other streptococci spp., Escherichia |
| | coli, Pseudomonas spp., Enterobacter spp., |
| | Proteus spp., Serratia spp., Neisseria gonorrhea, |
| | salmonella species, Mycobacterim tuberculosis, |
| | Hemophilus influenza |
| | parvovirus B19, rubella, hepatitis B |
| | Scedosporium prolificans |
| Meninges | Haemophilus influenzae, Neisseria meningitidis, |
| | Streptococcus pneumoniae, Streptococcus |
| | agalactiae, Listeria monocytogenes |
| | echoviruses, coxsackieviruses, other |
| | enteroviruses, mumps |
| Brain | Streptococcus spp. (including S. anginosus, S. |
| | constellatus, S. intermedius), Staphylococcus |
| | aureus, Bacteroides spp., Prevotella spp., |
| | Proteus spp., Escherichia coli, Klebsiella spp., |
| | Pseudomonas spp., Enterobacter spp., Borrelia |
| | burgdorferi |
| | coxsackieviruses, echoviruses, poliovirus, other |
| | enteroviruses, mumps, herpes simplex, varicella- |
| | zoster, flaviviruses, bunyaviruses |
| Spinal cord | Haemophilus influenzae, Neisseria meningitidis, |
| | Streptococcus pneumoniae, Streptococcus |
| | agalactiae, Listeria monocytogenes, Borrelia |
| | burgdorferi |
| | coxsackieviruses, echoviruses, poliovirus, other |
| | enteroviruses, mumps, herpes simplex, varicella- |
| | zoster, flaviviruses, bunyaviruses |
| Eye/Orbit | Staphylococcus aureus, Streptococcus |
| | pyogenes, Streptococcus pneumoniae, |
| | Streptococcus milleri, Escherichia coli, Bacillus |
| | cereus, Chlamydia trachomatis, Haemophilus |
| | influenza, Pseudomonas spp., Klebsiella spp., |
| | Treponema pallidum |
| | adenoviruses, herpes simplex, varicella-zoster, |
| | cytomegalovirus |
| Salivary | Staphylococcus aureus, viridans streptococci |
| glands | (e.g., Streptococcus salivarius, Streptococcus |
| | sanguis, Streptococcus mutans), |
| | Peptostreptococcus spp., Bacteroides spp., and |
| | other oral anaerobes |
| | mumps, influenza, enteroviruses, rabies |
| Oral | Prevotella melaninogenicus, anaerobic |
| | streptococci, viridans streptococci, Actinomyces |
| | spp., Peptostreptococcus spp., Bacteroides spp., |
| | and other oral anaerobes |
| | herpes simplex, coxsackieviruses, Epstein-Barr |
| Tonsil | Streptococcus pyogenes, Group C and G B- |
| | hemolytic streptococci |
| | rhinoviruses, influenza, coronavirus, adenovirus, |
| | parainfluenza, respiratory syncytial virus, herpes |
| | simplex |
| Sinus | Streptococcus pneumoniae, Haemophilus |
| | influenza, Moraxella catarrhalis, α-streptococci, |
| | anaerobic bacteria (e.g., Prevotella spp.), |
| | Staphylococcus aureus |
| | rhinoviruses, influenza, adenovirus, |
| | parainfluenza |
| Nasopharynx | Streptococcus pyogenes, Group C and G B- |
| | hemolytic streptococci |
| | rhinoviruses, influenza, coronavirus, adenovirus, |
| | parainfluenza, respiratory syncytial virus, herpes |
| | simplex |
| Thyroid | Staphylococcus aureus, Streptococcus |
| | pyogenes, Streptococcus pneumoniae |
| | mumps, influenza |
| Larynx | Mycoplasma pneumoniae, Chlamydophila |
| | pneumoniae, Streptococcus pyogenes |
| | rhinovirus, influenza, parainfluenza, adenovirus, |
| | corona virus, human metapneumovirus |
| Trachea | Mycoplasma pneumoniae |
| | parainfluenza, influenza, respiratory syncytial |
| | virus, adenovirus |
| Bronchi | Mycoplasma pneumoniae, Chlamydophila |
| | pneumoniae, Bordetella pertussis, Streptococcus |
| | pneumoniae, Haemophilus influenzae |
| | influenza, adenovirus, rhinovirus, coronavirus, |
| | parainfluenza, respiratory syncytial virus, human |
| | metapneumovirus, coxsackievirus |
| Lung | Streptococcus pneumoniae, Moraxella |
| | catarrhalis, Mycoplasma pneumoniae, Klebsiella |
| | pneumoniae, Haemophilus influenza |
| | influenza, adenovirus, respiratory syncytial virus, |
| | parainfluenza |
| Pleura | Staphylococcus aureus, Streptococcus |
| | pyogenes, Streptococcus pneumoniae, |
| | Haemophilus influenzae, Bacteroides fragilis, |
| | Prevotella spp., Fusobacterium nucleatum, |
| | peptostreptococcus spp., Mycobacterium |
| | tuberculosis |
| | influenza, adenovirus, respiratory syncytial virus, |
| | parainfluenza |
| Mediastinum | viridans streptococci, Peptococcus spp., |
| | Peptostreptococcus spp., Bacteroides spp., |
| | Fusobacterium spp. |
| | measles, rubella, Epstein-Barr, cytomegalovirus |
| Heart | Streptococcus spp. (including S. mitior, S. bovis, |
| | S. sanguis, S. mutans, S. anginosus), |
| | Enterococcus spp., Staphylococcus spp., |
| | Corynebacterium diptheriae, Clostridium |
| | perfringens, Neisseria meningitidis, Salmonella |
| | spp. |
| | enteroviruses, coxsackieviruses, echoviruses, |
| | poliovirus, adenovirus, mumps, rubeola, |
| | influenza |
| Esophagus | Actinomyces spp., Mycobacterium avium, |
| | Mycobacterium tuberculosis, Streptococcus spp. |
| | cytomegalovirus, herpes simplex, varicella-zoster |
| Stomach | Streptococcus pyogenes, Helicobacter pylori |
| | cytomegalovirus, herpes simplex, Epstein-Barr, |
| | rotaviruses, noroviruses, adenoviruses |
| Small bowel | Escherichia coli, Clostridium difficile, Bacteroides |
| | fragilis, Bacteroides vulgatus, Bacteroides |
| | thetaiotaomicron, Clostridium perfringens, |
| | Salmonella enteriditis, Yersinia enterocolitica, |
| | Shigella flexneri |
| | adenoviruses, astroviruses, caliciviruses, |
| | noroviruses, rotaviruses, cytomegalovirus |
| Colon/ | Escherichia coli, Clostridium difficile, Bacteroides |
| Rectum | fragilis, Bacteroides vulgatus, Bacteroides |
| | thetaiotaomicron, Clostridium perfringens, |
| | Salmonella enteriditis, Yersinia enterocolitica, |
| | Shigella flexneri |
| | adenoviruses, astroviruses, caliciviruses, |
| | noroviruses, rotaviruses, cytomegalovirus |
| Anus | Streptococcus pyogenes, Bacteroides spp., |
| | Fusobacterium spp., anaerobic streptococci, |
| | Clostridium spp., E. coli, Enterobacter spp., |
| | Pseudomonas aeruginosa, Treponema pallidum |
| | herpes simplex |
| Perineum | Escherichia coli, Klebsiella spp., Enterococcus |
| | spp., Bacteroides spp., Fusobacterium spp., |
| | Clostridium spp., Pseudomonas aeruginosa, |
| | anaerobic streptococci, Clostridium spp., E. coli, |
| | Enterobacter spp. |
| | herpes simplex |
| Liver | Escherichia coli, Klebsiella spp., Streptococcus |
| | (anginosus group), Enterococcus spp., other |
| | viridans streptococci, Bacteroides spp. |
| | hepatitis A, Epstein-Barr, herpes simplex, |
| | mumps, rubella, rubeola, varicella-zoster, |
| | coxsackieviruses, adenovirus |
| Gallbladder | Escherichia coli, Klebsiella spp., Enterobacter |
| | spp., enterococci, Bacteroides spp., |
| | Fusobacterium spp., Clostridium spp., |
| | Salmonella enteriditis, Yersinia enterocolitica, |
| | Shigella flexneri |
| Biliary tract | Escherichia coli, Klebsiella spp., Enterobacter |
| | spp., Enterococci spp., Bacteroides spp., |
| | Fusobacterium spp., Clostridium spp., |
| | Salmonella enteriditis, Yersinia enterocolitica, |
| | Shigella flexneri |
| | hepatitis A, Epstein-Barr, herpes simplex, |
| | mumps, rubella, rubeola, varicella-zoster, |
| | cocsackieviruses, adenovirus |
| Pancreas | Escherichia coli, Klebsiella spp., Enterococcus |
| | spp., Pseudomonas spp., Staphylococcal spp., |
| | Mycoplasma spp., Salmonella typhi, |
| | Leptospirosis spp., Legionella spp. |
| | mumps, coxsackievirus, hepatitis B, |
| | cytomegalovirus, herpes simplex 2, varicella- |
| | zoster |
| Spleen | Streptococcus spp., Staphylococcus spp., |
| | Salmonella spp., Pseudomonas spp., |
| | Escherichia coli, Enterococcus spp. |
| | Epstein-Barr, cytomegalovirus, adenovirus, |
| | measles, rubella, coxsackieviruses, varicella- |
| | zoster |
| Adrenal gland | Streptococcus spp., Staphylococcus spp., |
| | Salmonella spp., Pseudomonas spp., |
| | Escherichia coli, Enterococcus spp. |
| | varicella-zoster |
| Kidney | Escherichia coli, Proteus mirabilis, Proteus |
| | vulgatus, Providentia spp., Morganella spp., |
| | Enterococcus faecalis, Pseudomonas |
| | aeruginosa |
| | BK virus, mumps |
| Ureter | Escherichia coli, Proteus mirabilis, Proteus |
| | vulgatus, Providentia spp., Morganella spp., |
| | Enterococcus spp. |
| Bladder | Escherichia coli, Proteus mirabilis, Proteus |
| | vulgatus, Providentia spp., Morganella spp., |
| | Enterococcus faecalis, Corynebacterium jekeum |
| | adenovirus, cytomegalovirus |
| Peritoneum | Staphylococcus aureus, Streptococcus |
| | pyogenes, Streptococcus pneumoniae, |
| | Escherichia coli, Klebsiella spp., Proteus spp., |
| | Enterococci spp., Bacteroides fragilis, Prevotella |
| | melaninogenica, Peptococcus spp., |
| | Peptostreptococcus spp., Fusobacterium spp., |
| | Clostridium spp. |
| Retroperitoneal | Escherichia coli, Staphylococcus aureus |
| area | |
| Prostate | Escherichia coli, Klebsiella spp., Enterobacter |
| | spp., Proteus mirabilis, Enterococci spp., |
| | Pseudomonas spp., Corynebacterium spp., |
| | Neisseria gonorrhoeae |
| | herpes simplex |
| Testicle | Escherichia coli, Klebsiella pneumoniae, |
| | Pseudomonas aeruginosa, Staphylococcus spp., |
| | Streptococcus spp., Salmonella enteriditis |
| | mumps, coxsackievirus, lymphocytic |
| | choriomeningitis virus |
| Penis | Staphylococcus aureus, Streptococcus |
| | pyogenes, Neisseria gonorrhoeae, Treponema |
| | pallidum |
| | herpes simplex, human papillomavirus |
| Ovary/ | Neisseria gonorrhoeae, Chlamydia trachomatis, |
| Adnexae | Gardenerella vaginalis, Prevotella spp., |
| | Bacteroides spp., Peptococcus spp. |
| | Streptococcus spp., Escherichia coli |
| Uterus | Neisseria gonorrhoeae, Chlamydia trachomatis, |
| | Gardenerella vaginalis, Prevotella spp., |
| | Bacteroides spp., Peptococcus spp., |
| | Streptococcus spp., Escherichia coli |
| Cervix | Neisseria gonorrhoeae, Chlamydia trachomatis, |
| | Treponema pallidum |
| | herpes simplex |
| Vagina | Gardenerella vaginalis, Prevotella spp., |
| | Bacteroides spp., peptococci spp., Escherichia |
| | coli, Neisseria gonorrhoeae, Chlamydia |
| | Trachomatis, Treponema pallidum, |
| | herpes simplex |
| Vulva | Staphylococcus aureus, Streptococcus |
| | pyogenes, Treponema pallidum |
| | herpes simplex |

13. The use or method according to any one of claims 1 to 12, wherein the composition is for use by administration in an amount effective to modulate a biomarker during the perioperative period selected from the group consisting of MHCII, CD96, CD69, IFNγ.

14. The use or method according to any one of claims 1 to 13, wherein the composition is for use by administration in an amount effective in the perioperative period to modulate a biomarker that is: increased CD69 expression; reduced expression of CD96, increased expression of MHCII; increased myelopoiesis; increased neutrophil and/or monocyte cell counts; increased expression of CCR2 chemokine receptor expression on immune cells; and/or increased FNγ expression.

15. The use or method according to any one of claims 1 to 12, wherein the composition is for use in an amount effective to modulate a biomarker selected from the group consisting of PD1, PDL1, IP-10, MIG, RANTES, neutrophils, Ly6C monocytes, and NKG2D.

16. The use or method according to claim 15, wherein the composition is for use in an amount effective to down-regulate PD1 and/or PDL1 expression in cells present in the target tissue.

17. The use or method according to any one of claims 1 to 16, wherein the composition is for use by administration in an amount effective in the perioperative period to modulate a biomarker that is: upregulated cytotoxic granules (perforin, granzymes A & B); upregulated NKG2D; upregulated chemokines CXCL9 & CXCL10 (IP-10), upregulated IL-18; upregulated IL-1B, upregulated GM-CSF, upregulated NKG2D ligands; increased iNOS expression (as indicative of M1 macrophage polarization); upregulated CD86 (human M1 monocytes); upregulated GCSF; upregulated IL-2; upregulated IL-3; upregulate IL-6; upregulate IL-7;j upregulate IL-12(p70); upregulated IL-13;j upregulated IL-15, upregulated CXCL1; upregulated M-CSF; upregulated TNFα; downregulated PD-1, and/or downregulated PDL1, downregulate CD163 (human M2 monocytes).

18. The use or method of any one of claims 13 to 17, further comprising assaying a sample from the patient for the biomarker, optionally assaying during the perioperative period.

19. The use or method of any one of claims 1 to 18, further comprising monitoring an immune response in the patient by:
monitoring immune suppression by assaying a patient sample for an immune suppression biomarker that is: CTLA-4, KIR (Killer Inhibitory Receptors), CD43, arginase, IDO, TGFβ, CD155, myeloid suppressive cells (MDSCs), Treg cells (IL-10), soluble (cleaved) MICA/B, and/or soluble CD95, and/or
monitoring immune activation by assaying a patient sample for an immune activation biomarker that is: MICA/B and related NKG2D ligands expressed on human cells, activation of gd T cells, T-bet expression, IL-15, epigenetic changes associated with trained innate immunity, and/or metabolic changes (glycolysis>oxidative phosphorylation) of immune cells.

20. The use or method according to any one of claims 1 to 19, wherein the therapeutic vehicle is for administration at an administration site that is not the target tissue.

21. The use or method according to any one of claims 1 to 19, wherein the composition is for use by administration in an amount effective in the perioperative period to modulate the immune response so as to ameliorate post-surgical immune suppression.

22. The use or method according to claim 21, wherein the administration site is the skin or subcutaneous tissue.

23. The use or method according to any one of claims 1 to 22, wherein the therapeutic vehicle is formulated for systemic distribution of the PRR agonists following administration.

24. The use or method according to any one of claims 1 to 23, wherein the therapeutic vehicle is administered in a plurality of doses over a dosage duration, and the dosage duration is at least two weeks, optionally at least one week, optionally before and after the surgery date.

25. The use or method according to claim 24, wherein the doses are administered subcutaneously every day, or every other day, before and after the surgery date.

26. The use or method according to any one of claims 1 to 25, wherein the therapeutic vehicle comprises whole killed or attenuated Klebsiella pneumonia and the target tissue comprises: lung, brain, pancreas, prostate, testes or liver.

27. The use or method according to any one of claims 1 to 25, wherein the therapeutic vehicle comprises whole killed or attenuated E. coli and the target tissue comprises: colon, bowel, rectum, pancreas, kidney, bladder, prostate, testes, ovary or liver.

28. The use or method according to any one of claims 1 to 25, wherein the therapeutic vehicle comprises whole killed or attenuated Staphylococcus aureus and the target tissue comprises: skin, bone, brain or breast.

29. Use of an effective amount of an immunogenic composition to treat a cancer in a mammalian subject, wherein:
the cancer forms a tumor in a target tissue, and/or the cancer is characterized by a potential to metastasize to the target tissue;
the composition is for use in combination with surgical removal of the tumor on a surgery date;
the composition comprises a whole killed or attenuated microbial mammalian pathogen that is pathogenic in the target tissue; and,
the immunogenic composition is for use in a perioperative period that is within one month of the surgery date so as to modulate an immune response in the target tissue that is effective to treat residual disease and thereby treat the cancer.

30. A method for treating a cancer in a mammalian subject, wherein the cancer forms a tumor in a target tissue, and/or the cancer is characterized by a potential to metastasize to the target tissue, wherein the subject undergoes surgical removal of the tumor on a surgery date, the treatment comprising:
administering to the subject an effective amount of an immunogenic composition during a perioperative period that is within one month of the surgery date;
wherein the composition comprises a whole killed or attenuated microbial mammalian pathogen that is pathogenic in the target tissue; and,
the immunogenic composition is administered so as to modulate an immune response in the target tissue that is effective to treat residual disease and thereby treat the cancer.

31. An SSI delivery system comprising an SSI formulation for use in an effective amount to treat a cancer in a mammalian subject, wherein:
the cancer forms a tumor in a target tissue, and/or the cancer is characterized by a potential to metastasize to the target tissue;
the composition is for use in combination with surgical removal of the tumor on a surgery date, wherein surgical removal leaves a surgical wound;
the composition comprises a whole killed or attenuated microbial mammalian pathogen that is pathogenic in the target tissue; and,
the delivery system is for use on the surgery date applied to the surgical wound so as to mediate release of the composition and thereby modulate an immune response in the target tissue that is effective to treat residual disease and thereby treat the cancer.

32. The delivery system of claim 31, comprising a staged-release matrix encasing the mammalian pathogen, the staged-release matrix being adapted to release the mammalian pathogen from the matrix in a plurality of successive temporally separated dosing stages after the delivery system is applied to the surgical wound on the surgery date, with a therapeutically effective alliquote of mammalian pathogen being released at each dosing stage during a perioperative period that is within two months of the surgery date.

33. The delivery system of claim 31 or 32, wherein the staged-release matrix comprises a surface-eroding polymer.

34. The delivery system of claim 33, wherein the surface-eroding polymer comprises a polyanhydride and/or a poly(ortho ester).

35. The delivery system of claim 33, wherein the surface-eroding polymer comprises cellulose acetate phthalate complexed with Pluronic F-127.
